(19) United States
(12) Patent Application Publication
(10) Pub. No.: US 2026/0315489 A1
(43) Pub. Date: Oct. 15, 2026

(54) Oral Liposomal Formulations of Fasudil

(71) Applicant: Woolsey Pharmaceuticals, Inc., St. Petersburg, FL (US)

(72) Inventors: Qicai LIU, Short Hills, NJ (US); Vivek Gupta, Floral Park, NY (US); Mimansa Goyal, Jamaica, NY (US)

(73) Assignee: Woolsey Pharmaceuticals, Inc., St. Petersburg, FL (US)

(21) Appl. No.: 18/881,324

(22) PCT Filed: Jul. 5, 2023
(86) PCT No.: PCT/US2023/069631

§ 371 Date: Jan. 6, 2025

Related U.S. Application Data
(60) Provisional application No. 63/359,101, filed on Jul. 7, 2022.

(51) Int. Cl. A61K 31/551; A61K 9/00; A61K 9/1277; A61K 47/24; A61K 47/28

(52) U.S. Cl. CPC A61K 31/551; A61K 9/0053; A61K 9/1277; A61K 47/24; A61K 47/28

(57) ABSTRACT

An oral pharmaceutical composition comprising a liposome is provided. The composition can comprise a plurality of liposomes. The liposome can comprise fasudil, alone or in combination with another rho kinase inhibitors, a pharmaceutically acceptable salt thereof, a hydrate thereof, a prodrug thereof, a substituted derivative thereof, or a metabolite thereof, or any combination thereof. The liposome can comprise a phospholipid and cholesterol. The oral pharmaceutical composition can be formulated as any desired dosage form or combination of dosage forms. The liposome can comprise a coating. A method is provided that can comprise administering to a patient a liposomal composition in an amount sufficient to treat the neurogenerative disease or a symptom thereof. Methods of forming fasudil-containing liposomes formulated for oral administration are also provided, for example, coated liposomes.

### CROSS-REFERENCE TO RELATED APPLICATIONS

This international application claims the benefit of U.S. Provisional Patent Application No. 63/359,101, filed 7 Jul. 2022, which is incorporated by reference herein in its entirety.

### BACKGROUND

### Field

The present disclosure relates to oral liposomal formulations comprising a rho kinase inhibitor, for example, fasudil, as well as methods of producing and orally administering the same that can both diminish negative organoleptic effects to aid patient compliance and permit targeted delivery in the gastrointestinal tract for enhanced efficacy.

### Description of Related Art

Delivery of a drug intact to a target locus of absorption can present various challenges. Further, the elderly and particularly those suffering from neurodegenerative diseases can have difficulty adhering to a consistent drug regimen. Compliance can be further complicated for patients on a multi-drug regimen and those experiencing dysphagia. An enhanced aversion to certain tastes and mouthfeel as well as organoleptic perceptions can pose significant hurdles for such patients. When one attempts to address compliance issues associated with dysphagia by using a liquid dosage form, new problems can occur. An active pharmaceutical ingredient (API) having an unpleasant taste can be particularly unappealing when consumed in a liquid. Reducing or even eliminating such a taste can present significant obstacles to a formulator given the considerable number of variables relating both to the contents of an API formulation and the patient.

Solutions to these technical problems are provided by the present disclosure.

### BRIEF SUMMARY

An oral pharmaceutical composition comprising a liposome is provided in accordance with the present disclosure. The composition can comprise a plurality of liposomes. The liposome can comprise fasudil, alone or in combination with another rho kinase inhibitor, a pharmaceutically acceptable salt thereof, a hydrate thereof, a prodrug thereof, a substituted derivative thereof, or a metabolite thereof, or any combination thereof. The liposome can comprise a phospholipid and cholesterol. The liposome can comprise a monoacyl glyceride and cholesterol. The liposome can comprise a diacyl glyceride and cholesterol. An esterified fatty acid of the monoacyl or diacyl glyceride can comprise a saturated fatty acid, or an unsaturated fatty acid, or both. The esterified fatty acids of the diacyl glyceride can be the same or different. The oral pharmaceutical composition can be formulated as any desired dosage form or combination of dosage forms. For example, the oral pharmaceutical composition can be formulated as a solid dosage form, or a liquid dosage form, or both. The liposome can be sufficiently stable in an oral cavity to mask a bitter taste of the fasudil. The liposome can comprise a coating. The fasudil, a pharmaceutically acceptable salt thereof, a hydrate thereof, a prodrug thereof, a substituted derivative thereof, or a metabolite thereof, or any combination thereof can be present in the composition in an amount sufficient to treat a neurodegenerative disease.

A method is provided that can comprise administering to a patient a liposomal composition in an amount sufficient to treat the neurogenerative disease. The method can comprise diagnosing the neurodegenerative disease prior to administering the composition to the patient. The method can comprise ameliorating a symptom of the neurodegenerative disease. The symptom can comprise, for example, wandering. A method of treating a neurological disorder is provided. The method can comprise orally administering to a subject a composition comprising a liposome, the liposome comprising fasudil, a pharmaceutically acceptable salt thereof, a hydrate thereof, a prodrug thereof, a substituted derivative thereof, or a metabolite thereof, or any combination thereof. Methods of forming fasudil-containing liposomes are also provided, for example, coated liposomes.

### BRIEF DESCRIPTION OF THE DRAWINGS

For a further understanding of the nature, objects, and advantages of the present disclosure, reference can be made to the following detailed description, read in conjunction with the following drawings.

FIG. 1 is a graph of percent cumulative release of fasudil from liposomes over time in a FED simulated intestinal fluid.

### DETAILED DESCRIPTION

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

An oral pharmaceutical composition comprising a liposome is provided in accordance with the present disclosure. The liposome can comprise fasudil, a pharmaceutically acceptable salt thereof, a hydrate thereof, a prodrug thereof, a substituted derivative thereof, or a metabolite thereof, or any combination thereof. Fasudil can be replaced or combined with another rho kinase inhibitor. Any rho kinase inhibitor or combination of rho kinase inhibitors can be used. Reference to a rho kinase inhibitor is inclusive of compounds in the composition that can directly inhibit a rho kinase and compounds that become rho kinase inhibitors upon one or more modifications subsequent to administration, for example, a metabolic modification by one or more enzymes or other process. The rho kinase inhibitor can inhibit a rho kinase using any relevant mechanism. For example, the inhibition can be competitive, non-competitive, reversible, or reversible, or any combination thereof. The rho kinase inhibitor can be the only active pharmaceutical ingredient (API) in the composition or one or more additional APIs can be present in the composition. One or more additional APIs can be part of the liposome, or outside the liposome, or both.

The rho kinase inhibitor can inhibit one or more kinds of rho kinases. The rho kinase is generally a serine/threonine kinase. Examples of rho kinases include rho kinase 1 (rho-associated coiled-coil-containing protein kinase 1, ROCK1, p160-ROCK) and rho kinase 2 (rho-associated coiled-coil-containing protein kinase 2, ROCK2). The rho kinase inhibitor can inhibit ROCK1, or ROCK2, or both. The rho kinase inhibited can be a human rho kinase, a mammalian rho kinase, or a vertebrate rho kinase, or any combination thereof. The rho kinase inhibitor can inhibit a human rho kinase exclusively or also one or more rho kinase homologs in other species.

The rho kinase inhibitor can comprise a rho kinase inhibitor having a bitter taste, a pharmaceutically acceptable salt thereof, a hydrate thereof, a prodrug thereof, a substituted derivative thereof, or a metabolite thereof, or any combination thereof. The rho kinase inhibitor can comprise one or more of an isoquinoline ring, a sulphonyl group, and a homopiperazine ring, or one or more equivalent structural moieties. For example, the rho kinase inhibitor can comprise fasudil, a pharmaceutically acceptable salt thereof, a hydrate thereof, a prodrug thereof, a substituted derivative thereof, or a metabolite thereof, or any combination thereof. The rho kinase inhibitor can be in a crystalline form, or an amorphous form, or both. Any polymorph or combination of polymorphs of the rho kinase inhibitor can be used. A pseudopolymorph can be used.

Fasudil comprises an isoquinoline ring, connected via a sulphonyl group to a homopiperazine ring. Isoquinoline derivatives, such as fasudil, its active metabolite hydroxyfasudil (M3), dimethylfasudil and ripasudil, are a class of ROCK inhibitors than can be used. Other ROCK inhibitors are based on 4-aminopyridine structures, for example, Y-27632 (Yoshitomi Pharmaceutical). Other ROCK inhibitors that can be use include, for example, indazole, pyrimidine, pyrrolopyridine, pyrazole, benzimidazole, benzothiazole, benzathiophene, benzamide, aminofurazane, quinazoline, or a boron derivative, or any combination thereof. Some exemplary ROCK inhibitors are shown below:

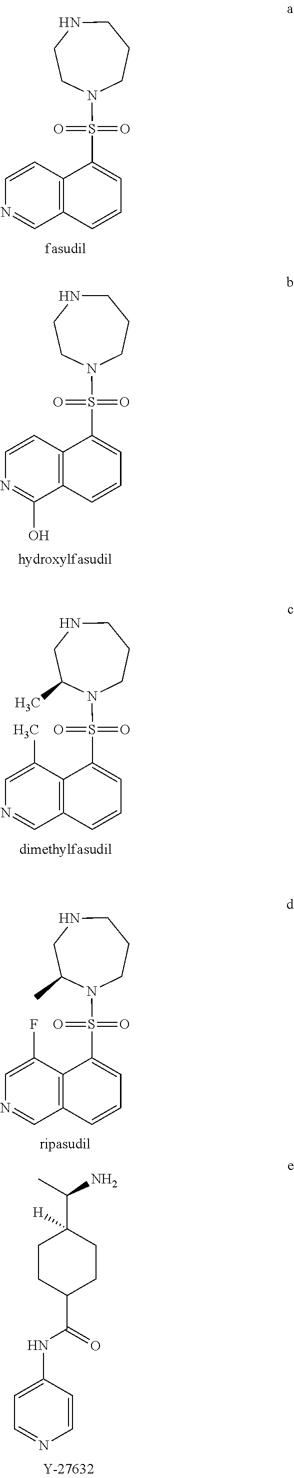

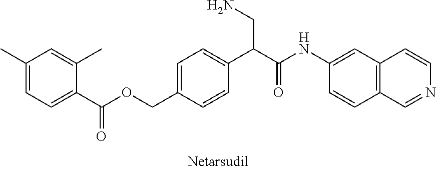 [SMILES: Cc1ccc(C(=O)OCc2ccc(C(CN)C(=O)Nc3ccc4cnccc4c3)cc2)c(C)c1]

Other examples of isoquinoline derived ROCK inhibitors include, for example, dimethylfasudil and ripasudil. Fasudil can exist as a free base or salt and can be in the form of a hydrate, such as a hemihydrate. Unless otherwise specified, references to a rho kinase inhibitor apply equally to the free acids or free bases, salts, hydrates, polymorphs and prodrug derivatives thereof. Fasudil can be, for example, in the form hexahydro-1-(5-isoquinolinesulfonyl)-1H-1,4-diazepine monohydrochloride hemihydrate:

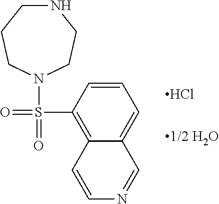 [SMILES: O=S(=O)(c1cccc2cnccc12)N1CCCNCC1 ; "·1/2 H2O" ; Cl]

In vivo, fasudil is subjected to hepatic metabolism to its active metabolite hydroxyfasudil (aka, M3). Fasudil is substantially converted in vivo to its 1-hydroxy (M3) metabolite. M3 exists as two tautomers, depicted below:

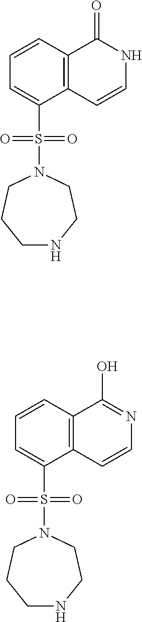 [SMILES: O=c1[nH]ccc2c(S(=O)(=O)N3CCCNCC3)cccc12 ; O=S(=O)(c1cccc2c(O)nccc12)N1CCCNCC1]

Rho kinase inhibitors, for example, fasudil, include pharmaceutically acceptable salts and hydrates. Salts can be formed via reaction with an inorganic, an organic acid, or both. Examples of suitable acids include hydrochloric acid, hydrobromide acid, hydriodic acid, sulfuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid, or any combination thereof.

The liposome can comprise a single lamellar vesicle. The liposome can comprise a phospholipid and cholesterol. The liposome can comprise a monoacyl glyceride and cholesterol. The liposome can comprise a monoacyl glyceride, a diacylglyceride, and cholesterol. The liposome can comprise a diacyl glyceride and cholesterol. The liposome can comprise the diacyl glyceride and cholesterol in a ratio of from about 0.1:1.0 to about 1.0:0.1, from about 0.25:1.0 to about 0.5:1.0, from about 0.5:1.0 to about 0.75:1.0, about 1:1, from about 1.0:0.1 to about 0.1:1.0, from about 0.75:1.0 to about 0.5:1.0, from about 0.25:1.0 to about 0.5:1.0, or any ratio therebetween, or any intervening range of ratios.

An esterified fatty acid of the diacyl glyceride can comprise a saturated fatty acid, or an unsaturated fatty acid, or both. The esterified fatty acids of the diacyl glyceride can be the same or different. The unsaturated fatty acid can comprise a monounsaturated fatty acid, or a polyunsaturated acid, or both. The esterified fatty acid of the diacyl glyceride can comprise, for example, capric acid, lauric acid, myristic acid, palmitic acid, stearic acid, arachidic acid, behenic acid, or lignoceric acid, or any combination thereof. The esterified fatty acid can have a carbon length of an even or odd number of carbons. The esterified fatty acid can have a carbon length of from about eight carbons to about 32 carbons, from about 10 carbons to about 28 carbons, from about 12 carbons to about 24 carbons, from about 14 carbons to about 22 carbons, from about 16 carbons to about 20 carbons, or any intervening carbon length, or any range of carbon lengths therebetween. The esterified fatty acid can have zero, one, two, three, four, five, six, or more carbon-carbon double bonds. The esterified fatty acid can comprise, for example, alpha-linolenic acid, gamma-linolenic acid, stearidonic acid, eicosenoic acid, eicosapentaenoic acid, cervonic acid, linoleic acid, linolelaidic acid, arachidonic acid, palmitoleic acid, vaccenic acid, paullinic acid, oleic acid, elaidic acid, gondonic acid, erucic acid, nervonic acid, or mead acid, or any combination thereof.

The diacyl glyceride can comprise a phospholipid. The phospholipid can comprise phosphatidylcholine, phosphatidylethanolamine, phosphatidylserine, phosphatidylglycerol, or phosphatidylinositol, or any combination thereof. The phospholipid can comprise, for example, dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoylphosphatidylethanolamine (DSPE), distearoylphosphatidylglycerol (DSPG), distearoylphosphatidylserine (DSPS), palmitoyloleoylphyosphatidylcholine (POPC), palmitoyloleoylphyosphatidylethanolamine (POPE), palmitoyloleoylphyosphatidylglycerol (POPG), palmitoyloleoylphyosphatidylserine (POPS), dioleoylphosphatidylethanolamine (DOPE), dioleoylphosphatidylserine (DOPS), dimyristoylphosphatidylcholine (DMPC), dimyristoylphosphatidylethanolamine (DMPE), dimyristoylphosphatidylglycerol (DMPG), or dimyristoylphosphatidylserine (DMPS), or any combination thereof. Phospholipids can be used as described, for example, in Drescher et al., “The Phospholipid Research Center: Current Research in Phospholipids and Their Use in Drug Delivery,” Pharmaceutics, 12, 1235 (2020), which is incorporated by reference herein in its entirety. Modified phospholipids can be used. For example, one or more phospholipids can be modified with polyethylene glycol to form pegylated-phospholipids. Any suitable cholesterol can be used. A standard cholesterol, a modified cholesterol, cholesterol-drug conjugate, or a cholesterol-containing polymer, or any combination thereof can be used. Cholesterol can be employed, for example, as described in Ruwizhi et al., “The Efficacy of Cholesterol-Based Carriers in Drug Delivery,” Molecules, 25, 4330 (2020), which is incorporated by reference herein in its entirety.

The oral pharmaceutical composition can be formulated as any desired dosage form or combination of dosage forms. For example, the oral pharmaceutical composition can be formulated as a solid dosage form, or a liquid dosage form, or both. The solid dosage form can comprise a tablet, or a capsule, or both. The tablet can comprise, for example, a buccal tablet, a sublingual tablet, a chewable tablet, an effervescent tablet, an orally disintegrating tablet, or a lozenge, or any combination thereof. A tablet can comprise a rho kinase inhibitor in a liposome as well as one or more excipients, for example, one or more of a diluent, a disintegrant, a lubricant, a colorant, a binder, a moisturizer, an absorbent, a glidant, and a solubilizer. The tablet can be a compressed tablet. The tablet can be uniform or non-uniform in composition. For example, the tablet can be a multilayer tablet. A capsule can comprise the rho kinase inhibitor in a liposome within a shell, for example, a shell comprising gelatin. A gelatin capsule can comprise a hard gelatin, or a soft gelatin, or both. A capsule can be considered as a solid dosage form in combination with a liquid dosage form of the present disclosure; the capsule surrounding a liquid composition of the liposomal rho kinase inhibitor. A capsule can comprise the same or similar excipients as tablets.

The oral pharmaceutical composition can be an aqueous composition. The aqueous composition can comprise, for example, a humectant, a chelator, an antioxidant, or a preservative, or any combination thereof. The aqueous composition can comprise, for example, a solution, a suspension, an emulsion, a gel, or a colloid, or any combination thereof. An aqueous composition can comprise a solution, a suspension, an emulsion, a gel, or a colloid, or any combination thereof. The aqueous composition can comprise a single phase or multiple phases. The aqueous composition can be an elixir. The aqueous composition can be a syrup. Water can be the sole solvent present in the aqueous composition. One or more additional solvents can be present in the aqueous composition. The additional solvent can comprise, for example, ethanol, glycerin, propylene glycol, or polyethylene glycol, or any combination thereof. One or more components of an aqueous composition can be in solution. One or more components of the same aqueous composition, for example, the liposomes, can be in suspension. A component in suspension can remain in suspension after mixing, shaking, vortexing, sonication, or other agitation for less than about 10 seconds, from about 10 seconds to about 10 years, from about 30 seconds to about 5 years, from about 1.0 minute to about 2.5 years, from about 2.0 minutes to about 1.5 years, from about 5.0 minutes to about 1.0 years, from about 10 minutes to about 8 months, from about 20 minutes to about 6 months, from about 30 minutes to about 3 months, from about 1.0 hour to about 1.0 month, from about 3.0 hours to about 30 days, from about 6.0 hours to about 3 weeks, from about 12 hours to about 2.0 weeks, from about 1.0 day to about 1.0 week, for about 3 days, or more than 10.0 years, or any period therebetween, or any range therebetween.

The aqueous composition can be buffered to resist or minimize change in pH. The aqueous composition can comprise a buffering agent. For example, the composition, aqueous or otherwise, can comprise a buffering agent. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, or ethyl alcohol, or any combination thereof. The aqueous composition can have a pH of less than about 4.0, greater than about 10.0, from about 4.0 to about 10.0, from about 5.0 to about 8.0, from about 5.5 to about 7.5, from about 6.0 to about 7.0, from about 6.2 to about 6.8, or from about 6.3 to about 6.5, or any value there between, or any range therebetween.

The aqueous composition can be formulated as an oil in water emulsion, a water in oil emulsion, or both. One or more components of an aqueous composition can be in the aqueous phase of an emulsion. One or more components of the same aqueous solution can be in the oil phase of the emulsion. The aqueous composition can comprise an emulsifier. The emulsifier can comprise one or more suspension agents. The emulsifier can comprise, for example, an anionic emulsifying wax, calcium stearate, a carbomer, a cetostaryl alcohol, cetyl alcohol, a cholesterol, a diethanolamine, an ethylene glycol palmitostearate, a glycerin monostearate, a glyceryl monooleate, hectorite, a lanolin, a lauric acid, a lecithin, linoleic acid, a triglyceride, a mineral oil, monobasic sodium phosphate, monoethanolamine, myristic acid, a nonionic emulsifying wax, octyldodecanol, oleic acid, oleyl alcohol, palmitic acid, polycarbophil, a polyoxyethylene alkyl ether, a polyoxyethylene castor oil derivative, a polyoxyethylene sorbitan fatty acid ester, a polyoxyethylene stearate, a saponite, a self-emulsifying glyceryl monostearate, sodium borate, sodium citrate dihydrate, sodium lauryl sulfate, a sorbitan ester, stearic acid, or triethanolamine, or any combination thereof.

The aqueous composition can be formulated as a gel. One or more gelling agents can be used, for example, aluminum stearate, calcium silicate, carbomers, carboxymethylcellulose sodium, carrageenan, chitosan, colloidal silicon dioxide, gelatin, glyceryl monooleate, glyceryl palmitostearate, guar gum, hydroxyethyl cellulose, microcrystalline cellulose, pectin, polyethylene alkyl ethers, polyethylene glycol, polyethylene oxide, polymethacrylates, propylene carbonate, sodium ascorbate, sodium alginate, sorbitol, urethane, or zinc acetate, or any combination thereof. A gel can have any desired viscosity. A gel or other composition described herein can be thixotropic.

The rho kinase inhibitor can be present at a concentration that allows for a reasonable volume of the aqueous composition per dose to be administered. For example, the rho kinase inhibitor can be present in an amount from about 0.1% w/v to about 10% w/v, from about 0.25% w/v to about 4.0% w/v, from about 0.33% w/v to about 3.0% w/v, from about 0.5% w/v to about 5.0% w/v, from about 1.0% w/v to about 2.5% w/v, from about 1.5% w/v to about 2.0% w/v of the aqueous composition, or any intervening percentage, or any intervening range. The taste threshold concentration can also correspond to one or more these percentages or ranges. A reasonable volume can be less than about 1.0 mL, from about 1.0 mL to about 1.0 L, from about 2.5 mL to about 500 mL, from about 4.0 mL to about 8 mL, from about 4.5 mL to about 6 mL, from about 5.0 mL to about 250 mL, from about 7.5 mL to about 100 mL, from about 10 mL to about 50 mL, from about 15 mL to about 30 mL, or greater than about 1.0 L, or any intervening volume, or any intervening volumetric range. A composition can be provided in a concentrate and then diluted before administration.

Each of one or more suspension agents can be present in the composition in any suitable amount. A suspension agent can be present, for example, in less than about 0.1% w/v, from about 0.1% w/v to about 50% w/v, from about 0.25% w/v to about 25% w/v, from about 1.0% w/v to about 20% w/v, from about 1.5% w/v to about 15% w/v, from about 2.0% w/v to about 10% w/v, from about 2.5% w/v to about 7.5% w/v, from about 3.5% w/v to about 6.0% w/v, from about 4.0% w/v to about 5.0% w/V, or more than about 25% w/v of the aqueous composition, or any intervening percentage, or any intervening range of percentages. For example, the first suspension agent can be present from about 0.5% w/v to about 2.0% w/v of the aqueous composition, and the second suspension agent can be present from about 0.1% w/v to about 1.0% w/v of the aqueous composition, or vice versa.

The composition can have any desired viscosity. For example, the viscosity of an aqueous composition can be less than about 1.0 centistoke (cSt), from about 1.0 cSt to about 1,000 cSt, from about 5.0 cSt to about 500 cSt, from about 10 cSt to about 400 cSt, from about 25 cSt to about 300 cSt, from about 40 cSt to about 250 cSt, from about 50 cSt to about 200 cSt, from about 60 cSt to about 180 cSt, from about 75 cSt to about 150 cSt, about 100 cSt, greater than about 1,000 cSt, or any intervening viscosity, or any intervening range. A chosen viscosity value or range can be achieved optionally with the presence of a viscosity modifier. A particular viscosity can be chosen to aid in compliance. An aqueous composition can be formulated in accordance with the International Dysphagia Diet Standardisation Initiative (IDDSI) for drink thickness on a scale of “0” for thin, “1” for slightly thick, “2” for mildly thick, “3” for moderately thick, or “4” for extremely thick as ascertained by an IDDSI flow test, all such tests being incorporated herein in their entireties. The IDDSI Framework and Detailed Level Definitions (July 2019) is incorporated herein by reference in its entirety.

A composition of the present disclosure can be packaged in any suitable form. For example, the composition can be packaged in a bottle or other liquid container in ready to use form. An optional cup or other volumetric dispenser can be supplied with the bottle. The cup can have a volume associated with a predetermined volumetric dose of the composition. The cup can optionally be graduated to allow for volumetric measurement. The cup can comprise one or more graduations or other markings indicative of one or more volumetric doses. The composition can be packaged as a dry concentrate, a liquid concentrate, or both. The composition can be packaged in separate admixture such that one or more components are segregated from one or more other components in two of more compartments of the packaging. A sachet, stick, capsule, or blister pack, or any combination thereof can be used, for example, in which each sachet, stick, capsule, or blister unit of a pack contains a dose of power, granules, or sprinkles, or any combination thereof, for example, for mixing with water alone or with one or more additional components to form the aqueous composition.

The liposome can be sufficiently stable in an oral cavity to mask a bitter taste of the fasudil. That is, the liposome can serve as a taste masking agent. For example, a liposome can help mask a bitter taste of fasudil or other rho kinase inhibitor by binding, entrapping, or encapsulating fasudil or the other rho kinase inhibitor. The fasudil or another rho kinase inhibitor can be located on the liposome, or in the liposome, or both. The fasudil or another rho kinase inhibitor can be on the liposomal surface, embedded in the liposome, contained within the liposome, or any combination thereof. The composition can further comprise one or more additional taste masking agent that masks the bitter taste. The taste masking agent can partially or fully mask the bitter taste of the rho kinase inhibitor. As used herein, a “taste masking agent” is a pharmaceutically acceptable agent that can mask the bitter taste of the rho kinase inhibitor, other unpleasant tastes present in the liquid composition, or any combination thereof. One or more taste masking agents can be present in the liquid composition. When more than one taste masking agent is present, the taste masking agents can act by the same or different mechanisms to mask the bitter taste of the rho kinase inhibitor. Multiple tasking masking agents can act synergistically. A taste masking agent can target the bitter taste of the rho kinase inhibitor directly, for example, by binding to the rho kinase inhibitor or blocking a bitterness taste receptor of a patient. A taste masking agent can indirectly target the bitter taste of the rho kinase inhibitor by distracting a patient's perception of the bitter taste, for example, using a sweetener, or a flavoring agent, or both. Taste masking can be partial or complete. The taste masking agent can be present in the composition in an amount sufficient to substantially neutralize the bitter taste. Substantial neutralization can essentially completely neutralize the bitter taste or the bitter taste can still be perceptible but with the composition being perceptible, but palatable, alone or in combination with other ingredients.

The taste masking agent can comprise a bitterness masker. As used herein, a “bitterness masker” refers to a taste masking agent configured to directly target or otherwise block the bitter taste of the rho kinase inhibitor, for example, by either binding the rho kinase inhibitor and preventing it from interacting with taste receptors or binding to bitterness receptors in the mouth and preventing them from binding to the rho kinase inhibitor. The bitterness masker can comprise a single type of molecule or a combination of different molecules that collectively mask the bitter taste of the rho kinase inhibitor. Different bitterness maskers can utilize the same, similar, or different mechanisms of action. The bitterness masker can mask bitter taste using any suitable mechanism. For example, the bitterness masker can bind or otherwise modify the rho kinase inhibitor. The bitterness masker can be, for example, an ion exchange resin such as a cation exchange resin, can bind the rho kinase inhibitor. Cyclodextrins, especially beta-cyclodextrins such as hydroxypropyl-β-cyclodextrin and sulfobutyl ether-β-cyclodextrin, represent another class of such bitterness maskers that can complex with the rho kinase inhibitor thereby preventing it from interacting with the taste receptors. Alginic acid, a linear ionic polymer, is another example of such a bitterness masker. The bitterness masker can bind less than 50% of the rho kinase inhibitor. The bitterness masker can bind at least 50%, at least 60%, at least 65%, at least 70%, at least 75%, at least 80%, at least 85%, at least 90%, at least 95%, at least 98%, at least 99%, or about 100% of the rho kinase inhibitor, or any intervening percentage, or any percentage therebetween. The bitterness masker can block or otherwise inhibit bitterness taste receptors; such a bitterness masker is referred to as a “bitter blocker.” Examples of bitter blockers include sodium acetate, sodium gluconate, adenosine 5′-monophosphate, and combinations thereof. A single kind of bitterness masker can be present in the composition. The composition can comprise a combination of two or more different kinds of bitterness maskers. Multiple kinds of bitterness maskers can act additively or synergistically in their ability to mask the bitter taste of the rho kinase inhibitor. Synergism can exist between a bitterness masker and one or more other taste masking agents.

The composition can comprise a plurality of fasudil-containing liposomes. The plurality of liposomes can comprise a fasudil-containing liposome described herein. At least 50% of liposomes, for example, in the plurality can comprise fasudil, a pharmaceutically acceptable salt thereof, a hydrate thereof, a prodrug thereof, a substituted derivative thereof, or a metabolite thereof, or any combination thereof. Less than about 1.0%, from about 1.0% to about 100% of liposomes, from about 5.0% to about 90%, from about 10% to about 75%, from about 10% to about 60%, from about 20% to about 50%, or any intervening percentage, or any range of percentages therebetween of liposomes can comprise fasudil. The concentration of fasudil, a pharmaceutically acceptable salt thereof, a hydrate thereof, a prodrug thereof, a substituted derivative thereof, or a metabolite thereof, or any combination thereof can be less than about 0.05 mg/mL, from about 0.05 mg/mL to about 50 mg/mL, from about 0.1 mg/mL to about 35 mg/mL, from about 1.0 mg/mL to about 25 mg/mL, from about 5.0 mg/mL to about 20 mg/mL, from about 10 mg/mL to about 15 mg/mL, or greater than 50 mg/mL, or any intervening concentration, or any range of concentration therebetween of the oral pharmaceutical composition. For example, the concentration of fasudil, a pharmaceutically acceptable salt thereof, a hydrate thereof, a prodrug thereof, a substituted derivative thereof, or a metabolite thereof, or any combination thereof is from about 5.0 mg/mL to about 25 mg/mL.

The polydispersity index (PDI) of the oral pharmaceutical composition can be less than about 1.0, less than about 0.75, less than about 0.6, less than about 0.5, less than about 0.3, or less than about 0.1. The plurality of liposomes comprises the liposome, and the mean diameter of the plurality of liposomes is from about 1.0 nm to about 500 μm, from about 5.0 nm to about 250 μm, from about 10 nm to about 100 μm, from about 25 nm to about 25 μm, from about 50 nm to about 5.0 μm, from about 100 nm to about 500 nm, or greater than about 500 μm, or any intervening mean diameter, or any range of mean diameters therebetween. A mean zeta value of the plurality of liposomes can be from about −50 mv to about −5 mv, from about −45 mv to about −10 mv, from about −40 mv to about −15 mv, from about −30 mv to about −20 mv, or any intervening zeta value, or any range of zeta values therebetween. The liposome or plurality of liposomes can comprise, for example, a small unilamellar vesicle, a large unilamellar vesicle, a giant unilamellar vesicle, a multilamellar vesicle, or multivesicular vesicle, or any combination thereof. Liposomes of the plurality can be present in the aqueous composition at a concentration of less than about 1.0% w/v, from about 1.0% w/v to about 80% w/v, from about 5.0% w/v to about 75% w/v, from about 10% w/v to about 60% w/v, from about 25% w/v to about 50% w/v, or greater than about 80% w/v, or any intervening percentage, or any range of percentages therebetween.

The composition can comprise any number of different suspension agents. For example, the composition can comprise, one, two, three, four, five, six, or more different suspension agents. The composition can comprise a first suspension agent and a second suspension agent. The aqueous composition can comprise a humectant, for example, ammonium alginate, cyclomethicone, glycerin, polydextrose, propylene glycol, sodium hyaluronate, sodium lactate, sorbitol, trehalose, triacetin, triethanolamine, or xylitol, or any combination thereof. The composition can comprise a chelator, an antioxidant, or a preservative, or any combination thereof. The pharmaceutical composition of the present disclosure can comprise a preservative. Exemplary preservatives include antioxidants, chelating agents, antimicrobial preservatives, antifungal preservatives, antiprotozoan preservatives, alcohol preservatives, acidic preservatives, and other preservatives. The preservative can be an antioxidant. The preservative can be a chelating agent. Exemplary antioxidants include alpha tocopherol, tocophersolan, citric acid monohydrate, erythorbic acid, ethyl oleate, fumaric acid, malic acid, phosphoric acid, phosphoric acid, tartaric acid, thymol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA) and salts and hydrates thereof (for example, sodium edetate, disodium edetate, trisodium edetate, calcium disodium edetate, dipotassium edetate, and the like), citric acid and salts and hydrates thereof (for example, citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, parabens, sorbic acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

The fasudil-containing liposome can comprise a coating. The coating can cover less than about 10%, from about 10% to 100%, from about 15% to about 99%, from about 20% to about 95%, from about 25% to about 90%, from about 30% to about 85%, from about 40% to about 75%, or from about 50% to about 60%, or any intervening percentage, or percentage range of the liposome or plurality of liposomes. The coating can be less than about 0.1 w/w %, from about 0.1 w/w % to about 50 w/w %, or greater than 10 w/w %, from about 1.0 w/w % to about 25 w/w %, from about 5.0 w/w % to about 15 w/w %, greater than about 50 w/w % of the fasudil-containing liposome, or any intervening percentage therebetween, or any intervening range of percentages. The coating can comprise an enteric coating, or a modified-release coating, or both. The coating can comprise a taste masking agent, for example, a bitterness masker, a sweetener, or a flavoring agent, or any combination thereof. The coating can comprise, on a weight/weight (w/w) basis relative to the fasudil-containing liposomes, less than about 10%, from about 10% to 100%, from about 15% to about 99%, from about 20% to about 95%, from about 25% to about 90%, from about 30% to about 85%, from about 40% to about 75%, or from about 50% to about 60%, or any intervening percentage, or percentage range of the fasudil-containing liposomes.

The oral pharmaceutical composition can comprise a coating covering the fasudil-containing liposome or plurality of fasudi-containing liposomes. The plurality of liposomes can be individually coated, or coated as a mass, or both. The oral pharmaceutical composition can comprise one or more coating agents, for example, acetyltributyl citrate, actetyltriethyl citrate, calcium carbonate, carboxymethylcellulose sodium, carnauba wax, cellulose acetate, cellulose acetate phthalate, cetyl alcohol, chitosan, ethylcellulose, fructose, gelatin, glycerin, glyceryl behenate, glyceryl palmitostearate, hydroxyethyl cellulose, hydroxyethylmethyl cellulose, hydroxylpropyl cellulose, hypromellose, hypromellose phthalate, isomalt, latex particles, liquid glucose, maltitol, maltodextrin, methylcellulose, microcrystalline wax, paraffin, poloxamer, polydextrose, polyethylene glycol, polyvinyl acetate, polyvinyl acetate phthalate, polyvinyl alcohol, potassium chloride, povidone, shellac, stearic acid, sucrose, tributyl citrate, triethyl citrate, vanillin, white wax, xylitol, yellow wax, or zein, or any combination thereof. The coating can comprise a cellulose ether, or a polymethacrylate, or both. The cellulose ether can comprise hydroxypropylmethyl cellulose (HPMC; hypromellose). The cellulose ether can comprise, for example, a methylcellulose (MC), an ethylcellulose (EC), a hydroxyethyl cellulose (HEC), a hydroxypropyl cellulose (HPC), a hydroxyethylmethyl cellulose (HEMC), a hydroxypropylmethyl cellulose (HPMC), a carboxymethyl cellulose (CMC), or a sodium carboxymethyl cellulose (NaCMC), or any combination thereof. The polymethacrylate can comprise a polymethacrylate having a methacrylic acid:methyl methacrylate ratio of 1:1, or 1:2, or both.

The fasudil, a pharmaceutically acceptable salt thereof, a hydrate thereof, a prodrug thereof, a substituted derivative thereof, or a metabolite thereof, or any combination thereof can be present in the composition in an amount sufficient to treat a neurodegenerative disease. The neurodegenerative disease can comprise Alzheimer's disease, vascular dementia, amyotrophic lateral sclerosis (ALS), Parkinson's disease, Huntington's disease, multiple sclerosis, progressive supranuclear palsy (PSP), or corticobasal syndrome (CBS), or any combination thereof. A method is provided that can comprise administering to a patient a liposomal composition in an amount sufficient to treat the neurogenerative disease. The method can comprise diagnosing the neurodegenerative disease prior to administering the composition to the patient. The method can comprise ameliorating a symptom of the neurodegenerative disease. The symptom can comprise, for example, wandering. A method of treating a neurological disorder is provided. The method can comprise orally administering to a subject a composition comprising a liposome, the liposome comprising fasudil, a pharmaceutically acceptable salt thereof, a hydrate thereof, a prodrug thereof, a substituted derivative thereof, or a metabolite thereof, or any combination thereof.

A method of treating a neurodegenerative disease is provided, the method comprising administering to a patient a composition of the present disclosure in an amount sufficient to treat the neurogenerative disease. For example, the amount of rho kinase inhibitor per dose can be less than about 0.5 mg, from about 0.5 mg to about 250 mg, from about 5.0 mg to about 225 mg, from about 10 mg to about 200 mg, from about 20 mg to about 180 mg, from about 25 mg to about 170 mg, from about 30 mg to about 160 mg, from about 40 mg to about 150 mg, from about 50 mg to about 140 mg, from about 60 mg to about 120 mg, from about 75 mg to about 100 mg, or more than 250 mg, or any amount therebetween, or any range therebetween. These amounts can refer to the rho kinase inhibitor, a pharmaceutically acceptable salt thereof, a hydrate thereof, a prodrug thereof, a substituted derivative thereof, or a metabolite thereof, or any combination thereof. Some examples of doses per day are set forth in Table 1 with respect to various therapeutic targets. Doses outside, within, or overlapping these ranges can also be employed. Doses can be adjusted based on the weight, health, and other factors of a specific patient or a group of patients.

Fasudil hydrochloride hemihydrate can be substituted by a molar equivalent of another salt, hydrate, prodrug, or active metabolite of fasudil or other rho kinase inhibitor. Dosing can be performed once a day, twice a day, three times a day, four times a day, every two days, every three days, every week, every other week, once a month, less frequently, more frequently, or any frequency therebetween, or any range of frequencies therebetween.

TABLE 1

| | Exemplary Dose | | Exemplary Dose | |
|---|---|---|---|---|
| Indication | Ranges | | and/or Dose Range | |
| Wandering in | 70 to 120 | mg/day | 90 | mg/day |
| Alzheimer's | 80 to 140 | mg/day | | |
| | 100 to 180 | mg/day | | |
| Pseudobulbar Affect | 70 to 140 | mg/day | 90 | mg/day |
| Frontotemporal | 60 to 120 | mg/day | 60 to 120 | mg/day |
| dementia | 70 to 140 | mg/day | 90 to 120 | mg/day |
| | 90 to 180 | mg/day | | |
| Dementia with Lewy | 70 to 120 | mg/day | 90 | mg/day |
| Bodies | 80 to 140 | mg/day | | |
| | 100 to 180 | mg/day | | |
| Alzheimer's | 70 to 140 | mg/day | 90 | mg/day |
| Vascular dementia | 70 to 180 | mg/day | 90 to 140 | mg/day |
| Neurodevelopmental | 10 to 200 | mg/day | 0.5 to 3 | mg/kg/day |
| diseases | 70 to 150 | mg/day | | |
| | 80 to 120 | mg/day | | |
| Proteinopathies | 70 to 180 | mg/day | 80 to 150 | mg/day |
| | 80 to 160 | mg/day | | |
| | 100 to 130 | mg/day | | |
| Age-related | 70 to 180 | mg/day | 80 to 150 | mg/day |
| cognitive decline | 80 to 160 | mg/day | | |
| | 100 to 130 | mg/day | | |
| Low-dose age related | 16 to 60 | mg/day | 35 to 50 | mg/day |
| cognitive decline | 20 to 40 | mg/day | | |
| | 24 to 30 | mg/day | | |
| 4-Repeat tauopathies | 70 to 180 | mg/day | 80 to 150 | mg/day |
| | 80 to 160 | mg/day | | |
| | 100 to 130 | mg/day | | |
| Low-dose dementia- | 2.5 to 30 | mg/day | 25 | mg/day |
| associated wandering | 5.0 to 25 | mg/day | | |
| | 10 to 20 | mg/day | | |
| Schizophrenia L (with | 70 to 180 | mg/day | 100 to 180 | mg/day |
| ARHGAP10 mutation) and | 80 to 160 | mg/day | | |
| other mental disorders | 100 to 130 | mg/day | | |
| Neurodevelopmental | 70 to 180 | mg/day | 70 to 120 | mg/day |
| disorders | 80 to 160 | mg/day | | |
| | 100 to 130 | mg/day | | |
| L-DOPA-induced | 70 to 180 | mg/day | 80 to 120 | mg/day |
| dyskinesia | | | | |
| Sporadic ALS | 90 to 180 | mg/day | 120 to 170 | mg/day |
| | 100 to 150 | mg/day | | |
| | ER 90-240 | | | |
| Anxiety/Agitation in | 90 to 180 | mg/day | 120 to 160 | mg/day |
| Alzheimer's and | 100 to 150 | mg/day | | |
| other dementias | 110 to 130 | mg/day | | |

Dosing can be modified and customized for a particular patient situation. For example, dosing can be modified for a patient with renal impairment. Dosing of fasudil for a patient with mild to moderate renal impairment can be reduced to about 5.0 to about 25 mg per day to about 2.5 mg to about 15 mg per day. An individual dose of fasudil can be used. Dosing can be reduced for a patient having serum creatinine (SCr)>2 and/or an increase in SCr>1.5× from baseline, and/or a decrease in eGFR>25% from baseline. Liver health and metabolic enzyme alleles can be considered in dosing. For example, dosing can be modified depending on whether a patient is fast or slow metabolizer of fasudil or another rho kinase inhibitor. Patient size can be factored when using creatinine-based estimates of renal function. Units of drug clearance of volume/time (mL/min) can be used, and units of estimated GFR for chronic renal disease of volume/time/standard size (mL/min/1.73 m²) can be used. Doses can be adjusted down (for example, to about 2.5 mg to about 15 mg per day) for smaller patients and up for larger (for example, up to 30 mg per day) for obese patients. A smaller male could be about 160 pounds or less. A smaller female patient could weigh about 130 pounds or less. Patients having a Body Mass Index (BMI) of 30 and higher can be considered obese.

Older patients can be administered a lower dose at initiation, with a gradual increase to the recommended dose after days or weeks. Older patients can be administered lower doses for the duration of treatment. The aged population can comprise the “young old” who can be from about 65 years old to about 74 years old, the “old old” who can be from about 75 years old to about 84 years old, and the “frail elderly” who can be 85 years old and older. For example, a starting dose of 10 mg per day for two weeks, followed by 20 mg per day for 4 weeks, then by 25 mg per day can be administered. Titration can be performed, for example, up to 30 mg per day. Methods of administering compositions can be continued for any suitable duration, for example, at least one day, for up to 30 days, for up to 60 days, or for up to 90 days, for at least six months, or for more than 90 days. Duration of treatment can depend on the patient's condition and response to treatment. A method can further comprise diagnosing a neurodegenerative disease in the patient prior to administering the composition to the patient. Treating the neurogenerative disease can comprise ameliorating a symptom of the neurodegenerative disease. The administration can comprise self-administration. The method can comprise tracking compliance of the administration.

A composition, or method, or both of the present disclosure can combine one or more rho kinase inhibitors, for example, fasudil, with other compounds used to treat dementia or other symptoms of dementia. They can be administered in combination, in a single dosage form, in separate dosage forms, through the same route of administration, through different routes of administration, in a common dosing regimen, or in independent dosing regimens, or any combination thereof. A composition can comprise, for example, fasudil in combination with one or other active pharmaceutical ingredients (APIs) suitable for treating cortical dementia, for example, cholinesterase inhibitors and NMDA receptor antagonists. A cholinesterase inhibitor can be selected from one or more of donepezil, rivastigmine, and galantamine. Exemplary doses of the cholinesterase inhibitors can comprise from about 3.0 mg to about 25 mg per day, or from about 6.0 mg to about 12 mg per day. The NMDA receptor antagonist can be memantine. For example, memantine can be administered at a dose of from about 5.0 to about 28 mg per day, or from about 15 mg to about 20 mg per day. A co-administered API can be, for example, a combination of donepezil and memantine at a dose of about 28 mg memantine and about 10 mg donepezil. A combination of fasudil with one or more cholinesterase inhibitors can be administered to wandering patients with proteinopathy-associated cortical dementia. A combination of fasudil with cholinesterase inhibitors can be administered to wandering patients with mixed dementia. Administration of a combination of fasudil with cholinesterase inhibitors can be excluded for wandering patients with only vascular cortical dementia. Dextromethorphan hydrobromide is another NMDA receptor antagonist that also has activity as a sigma-1 receptor agonist. Marketed in combination quinidine sulfate (a CYP450 2D6 inhibitor), the product Nudexta is indicated for the treatment of pseudobulbar affect, which occurs in many forms of dementia. Such APIs and medications can be administered in combination with rho kinase inhibitors such as fasudil. A patient treated with a composition described herein can be treated for depression, for example, a patient being treated with an anti-depressant such as citalopram or escitalopram.

A method is provided by the present disclosure comprising administering to a patient a composition of the disclosure in an amount sufficient to ameliorate a symptom of a neurodegenerative disease. For example, the symptom can comprise wandering. Wandering can comprise such behaviors as excess movement and pacing. A patient can be assessed by evaluating a physical parameter, a psychological parameter, or both. Assessment can be made through a verbal neurological test, for example, a written neurological test, or an oral neurological test, or both. Assessment can be made through observation of a patient's physical behavior, or psychological behavior, or both. Assessment can be made through imaging, for example, brain computer aided tomography (CAT), brain magnetic resonance imaging (MRI), brain diffuse tensor imaging (DTI), or brain positron emission tomography (PET), or any combination thereof. Imaging can evaluate any suitable feature, for example, blood flow, brain size, brain morphology, brain tissue, or physical feature of the brain, or any combination thereof with respect to the whole brain or part thereof. A physical feature can comprise, for example, a plaque, a tangle, a body, or an inclusion, or any combination thereof. Exemplary relevant brain parts include ventricles, the hippocampus, the frontal operculum, pre-central gyri, the midbrain, the pons and superior cerebellar peduncle, white matter tract integrity, a slant tract, the superior longitudinal fasciculus, the superior cerebellar peduncle, or the substantia nigra, any combination thereof. Assessment can be made by evaluating one or more biomarker, for example, from patient urine, patient blood, or patent cerebrospinal fluid, or any combination thereof. A biomarker can comprise, for example, a nucleic acid, a protein, a polysaccharide, a lipid, a metal, a salt, a neurotransmitter, an enzyme, a transcription factor, a receptor, a ligand, a vitamin, a mineral, or a metabolite, or any combination thereof. A biomarker can be evaluated with respect to a level or change in concentration, expression, distribution, structure, folding, conformation, molecular interaction, or chemical modification, or any combination thereof. Examples of neurological biomarkers comprise amyloid beta protein, alpha synuclein, a tau protein, phosphorylated tau, neurofilament light chain (NfL), total tau fragment levels, a fused in sarcoma (FUS) protein, a TAR DNA-binding protein 43 (TDP-43), a prion, or a neurotransmitter such as dopamine, or any combination thereof. Assessment can be made through any suitable protocol, for example, a Global Impression of Wandering (GIW), a Revised Algase Wandering Scale-Community Version (RAWS-CV), a Mini Mental State Examination (MMSE), a Neuropsychiatric Inventory-Questionnaire (NPI-Q), a Cohen-Mansfield Agitation Inventory-Community Version (CMAI-C), a Center for Neurological Study-Lability Scale (CNS-LS), or a Zarit Burden Interview (ZBI), or any combination thereof.

Patients treatable with compositions disclosed herein can score poorly on cognitive scales, such as the mini mental state exam (MMSE). A threshold of ≤23 on the MMSE can be set for dementia, with score of ≤15 representing severe dementia. Patients can be treated with an MMSE score ≤23, including moderately demented patients having an MMSE score of 16-23 and severe patients having an MMSE score ≤15. Once a patient has an MMSE score of less than 9, they can develop problems walking and treatment of patients with an MMSE less than 5 can be omitted. Once the MMSE falls below 15, the Severe Impairment Battery (SIB) can be a useful assessment. Treatment using compositions disclosed herein can result in improved cognitive functioning. Patients can show improvement on the MMSE and the SIB of at least 3 points during the early stages of treatment. Decline in cognition can be slowed relative to control patients.

Wandering and other symptoms can be treated by the compositions and methods of the present disclosure. Wandering can be characterized by two domains. The first domain can be movement, generally in the form of ambulation unless the patient is disabled and, for example, confined to a wheelchair. The second domain can be problematic behavior, for example in the form of boundary transgressions, or wayfinding problems, or both. Such behavior can be reflected in the movement itself, such as pacing or lapping behavior. The behavior can involve inappropriately following a caregiver. One common problematic behavior is attempted escape or elopement. A certain quantity of movement can also be considered the problematic behavior. A normal person can be in motion approximately 10% of their waking hours and so movement beyond this threshold amount can be considered problematic behavior. A patient can be considered to suffer from wandering when in motion for at least 20% of their waking hours, or more than 30% of their waking hours. As a patient spends more time in motion, the behavior can become particularly problematic because they risk exhaustion and, therefore, falling and serious injury. Wandering patients can be in motion more that 40% or 50% of their waking hours and some more than 60%, 70% or even 80%.

Wandering and other symptoms can be persistent or sporadic and the present compositions can be used to treat either population. Persistent wanderers can exhibit excessive movement nearly every day, typically at least 4 to 5 days per week. Sporadic wanderers generally do not exhibit excessive movement, but rather they are generally sedentary with occasional movement, typically associated with elopement, boundary transgressions, escape or wayfinding defects. Sporadic wanders can exhibit the behavior as infrequently as monthly or as frequently as 2, 3 or even 4, 5, 6, or more times per week. Unlike the persistent wanderer, the sporadic wonderer does not spend an abnormally high amount of time in motion. Patients treated can wander due to dementia of any form and not display a wayfinding defect; such a patient can be a persistent or a sporadic wanderer.

Compositions of the present disclosure can be used in the treatment of wandering or other symptoms in patients with vascular dementia (VaD). A composition can be used to treat mixed dementia, having pathologies not exclusively attributable to VaD and overlapping with other dementias, such as Alzheimer's dementia (AD). Any type or subtype of VaD can be treated, for example, VaD stemming from the various etiologies and pathologies associated with the same. Compositions of the present disclosure can be used to reduce or eliminate various symptoms and mood disorders associated with VaD.

Two main subtypes vascular dementia are i) large cortical infarction or multi-infarct dementia (MID) and ii) small vessel disease-related dementia or subcortical vascular dementia. Subcortical vascular dementia can be caused by disruption of the vasculature in the subcortical white matter-rich areas of the brain. The International Classification of Diseases (10th revision) (ICD-10) criteria for vascular explicitly identifies subcortical vascular dementia as a subgroup. Subcortical vascular dementia can incorporate the old entities “lacunar state” and “Binswanger disease” and relate to small vessel disease and hypoperfusion resulting in focal and diffuse ischemic white matter lesion and incomplete ischemic injury. Most dementia patients (mostly non-VaD patients) can suffer from the first type, affecting the cortical regions of the brain, and present with different defects that result from very different pathophysiological processes.

Large vessel cortical strokes and subcortical small vessel disease can tend to produce different kinds of deficits. Characteristic symptoms of subcortical dementia can include forgetfulness, slowing of thought processes, mild intellectual impairment, apathy, inertia, depression (sometimes with irritability), loss of recall ability, and the inability to manipulate knowledge. Subcortical dementia patients can have mood disorders. Other behavioral abnormalities like repetitive and compulsive behavior can occur in some patients suffering from subcortical dementia. Sub-cortical dementia presentation can be more subtle and temporally progressive, often described as defects in executive function in sub-cortical dementia. These can include deficits in speed and “strategic” processing (for example, attention, planning, and monitoring) in tasks such as memory tasks. Cortical vascular dementia can be associated with aphasia, apraxia and amnesia.

The American Psychiatric Association differentiates between mild and major neurocognitive impairment. Mild neurocognitive impairment can be defined as a cognitive decline one to two standard deviations from normal on formal cognitive testing that does not interfere with independence and is not due to delirium or other medical or psychiatric disorder. Major neurocognitive impairment can be defined as a cognitive decline two standard deviations or more from normal on formal cognitive testing that does interferes with independence and is not due to delirium or other medical or psychiatric disorder. VaD patients can have a major neurocognitive impairment according to these criteria, such that the impairment interferes with their independence. Impairment of independence can be assessed using a scale that measures activities of daily living (ADL), including scales like the Barthel Index and the Alzheimer's Disease Cooperative Study ADL inventory. Patients treatable with compositions according to the disclosure can have restricted independence in that they are residents in an assisted living or a memory care facility and are not community- or home-dwelling due to their condition.

Diagnostic and Statistical Manual of Mental Disorders Fifth Edition (DSM-V) provides a useful framework for the identification of patients treatable with compositions according to the disclosure. The DSM-V provides definitions of dementia syndrome. Dementia syndrome comprises objective cognitive or behavioral impairment in at least two of the following: memory; reasoning and handling complex tasks; visuospatial abilities; language functions; and personality, behavior, or comportment. Dementia syndrome can also comprise a decline from previous level of functioning and a functional impairment. VaD dementia can be precipitated by a cardiovascular event, such as an ischemic or hemorrhagic stroke, or a chronic cardiovascular condition, such as Binswanger's disease or lucunar dementia. VaD patients can be readily identified using the criteria of the National Institute of Neurological Disorders and Stroke (NINDS) and the Association Internationale pour la Recherche et l'Enseignement en Neurosciences (AIREN) (the NINDS-AIREN criteria). The NINDS-AIREN criteria comprise confirmation of vascular pathology using imaging. Patients identified according to the NINDS-AIREN criteria can be specifically included.

Compositions of the present disclosure can be used to treat wandering or other symptoms in diseases with an underlying proteinopathy, including Huntington's disease, autism spectrum disorder, Down syndrome, and dementia. Proteinopathy-associated dementia can result, for example, from Alzheimer's Disease (AD), Dementia with Lewy Bodies (DLB), Frontotemporal Dementia (FTD), head injuries, normal pressure hydrocephalus, Creutzfeldt-Jakob disease, amyotrophic lateral sclerosis, or Parkinson's disease, or any combination thereof. Where wandering occurs in a neurological condition associated with a proteinopathy, such a condition can be considered to be proteinopathy-associated wandering and treatable with compositions according to the disclosure.

A common underlying cause of proteinopathy-associated wandering is generally dementia. Dementia can comprise a set of symptoms related to a decline in memory and/or cognitive skills of such severity to adversely impact activities of daily living. Recognizing this, the definitive classification of dementia can be based on the underlying neuropathology. The primary neurodegenerative dementias AD, DLB, Parkinson's Disease dementia, FTD, and dementia associated with prion diseases (like CJD) can be characterized by progressive proteinopathy, which is an accumulation of misfolded proteins that lead to neuronal loss, neuroinflammation and glial reaction. Neurodegenerative dementias can be differentiated by the location and nature of misfolded protein accumulation. Thus, an understanding of the applicable underlying pathology of the dementia can be used to inform rational treatment of what are considered different underlying conditions.

Compositions of the present disclosure can be used to treat wandering or other symptoms associated with an underlying proteinopathy. Proteinopathy-associated dementia can refer to any form of dementia in which proteinopathy is considered to be part of the pathophysiology of the dementia. Proteinopathy is associated with lesions that comprise aggregates or deposits of protein that are generally not present in normal tissues. Alzheimer's disease, for instance, is associated with amyloid plaques, consisting of aggregates of Abeta4, and fibrillary tangles, consisting of deposits of phosphorylated tau. Frontotemporal dementia is associated with deposits of tau, TDP-43 and/or FUS. Pure vascular dementia can be independent of proteinopathy. Both or either of Alzheimer's disease and frontotemporal dementia can be included in treatment regimens. A pure vascular dementia can be excluded from treatment regimens. Compositions can be used to treat wandering in patients with conditions associated with abnormal deposits of Huntingtin protein (HTT), FUS, TDP-43, tau, amyloid-beta (for example, amyloid-beta42), optineurin, ubiquitin 2, superoxide dismutase 1, neurogenic locus notch homolog protein 3 (NOTCH3), or alpha-synuclein, or any combination thereof. Such deposits and their included proteins can be used as biomarkers.

Compositions can be used to treat wandering or other symptoms in male or female human patients suspected of having Parkinson's Disease with compositions of the disclosure. Synucleinopathies, which are also called alpha-synucleinopathies, are degenerative neurological diseases characterized by abnormal accumulation of aggregates of alpha-synuclein protein in neuros, nerve fibers, or glial cells. Three main types of synucleinopathy include Parkinson's disease (PD), dementia with Lewy bodies (DLB), and multiple system atrophy (MSA). PD patients can develop motor symptoms, namely tremor, slowness of movement, rigidity, and postural instability, as well as non-motor symptoms, which include autonomic dysfunction, neuropsychiatric problems (for example, mood, cognition, behavior, or thought alterations), affected senses, and sleep difficulties, including wandering.

Compositions can be used to treat wandering or other symptoms in male or female human patients suspected of having dementia Lewy bodies (DLB) with compositions according to the disclosure. Lewy bodies are abnormal clumps of protein (namely alpha-synuclein proteins, but can also include tau proteins) that develop in neural cells affected by PD, Lewy body dementias (DLB), and other disorders. DLB, in turn, is a progressive and degenerative neurological dementia, and, like PD, is a synucleinopathy. Because damage in the brain can be widespread, many domains of functioning can be affected. DLB can be distinguished from AD in that short-term memory impairment can be an early and prominent feature in AD, whereas memory impairment typically can occur later in DLB. Additionally, severe atrophy of the hippocampus can be more typical of AD than DLB. Patients with DLB can experience changes in sleep (including wandering), behavior, cognition, movement, and autonomic bodily functions.

Compositions can be used to treat wandering or other symptoms in human patients suspected of having Huntington's Disease with compositions of the disclosure. Huntington's disease (HD) is associated with aggregates of the huntingtin protein (Htt) and is generally inherited. Individuals have two copies of the huntingtin gene (HTT), which contains a trinucleotide repeat of cytosine-adenine-guanine (CAG). Individuals with 35 or fewer CAG repeats in the HTT gene are not affected by HD, individuals with 36-39 CAG repeats might or might not be affected by HD, and individuals with 40 or more CAG repeats are usually affected by HD. Htt aggregates accumulate to form inclusion bodies within cells and disrupt neuronal function and can be cytotoxic.

Compositions can be used to treat wandering or other symptoms in male or female human patients suspected of having diseases with an underlying 4R tauopathy with compositions according to the disclosure. Preferred 4R tauopathies include PSP, CBD, AGD, GGT, which are neuropathologically characterized by accumulation of phosphorylated 4R tau aggregates in neurons and certain glial cells. Depending on the type and location of the specific pathology, 4R tauopathies can manifest as a number of different clinical syndromes. There can be significant overlap between PSP and CBS (the clinical manifestation of CBD) and there can also be significant similarity in the neuropathology, suggesting that they are highly related, if not manifestations of the same condition. A conclusive diagnosis of 4R tauopathies can be made by examining the brain tissue by autopsy. Patients treatable with compositions according to the disclosure can be considered to have “probable” or “possible” disease on this basis. These patients can be considered to have a 4R tauopathy, even though it has not been confirmed pathologically. Treatment of wandering in a patient with a 4-R tauopathy can be considered the treatment of a patient with probable or possible disease, as well as someone with the confirmed pathology should that become possible in the future without an autopsy, using, for example, imaging or biomarkers. Similarly, co-pathologies can also be present in patients treatable with compositions described herein. These include Alzheimer's disease-related pathology (including cerebral amyloid angiopathy), Lewy-related and transactive response DNA-binding protein 43 and other proteinopathies. Cerebrovascular disease, including small vessel disease is a common co-pathology.

Diagnostic criteria for progressive supranuclear palsy (PSP) can be followed. Probable PSP refers to a patient with confirmed diagnosis. Various clinical manifestations of probable PSP can be discerned, depending on the predominant clinical features. These include, for example, PSP with Richardson's syndrome (PSP-RS); PSP with progressive gait freezing (PSP-PGF); PSP with predominant parkinsonism (PSP-P); PSP with predominant frontal presentation (PSP-F); PSP with predominant ocular motor dysfunction (PSP-OM); PSP with predominant speech/language disorder (PSP-SL); PSP with predominant CBS (PSP-CBS); PSP with predominant postural instability (PSP-PI).

The clinical features of PSP can be divided into the following functional domains: ocular motor dysfunction, postural instability, akinesia and cognitive dysfunction. The mostly highly correlated (“Level 1”) clinical features of PSP can be vertical supranuclear gaze palsy, repeated unprovoked falls within 3 years, progressive gait freezing within 3 years, and speech/language disorder (nonfluent/agrammatic variant of primary progressive aphasia or progressive apraxia of speech). Also highly correlated with PSP (“Level 2” clinical features) can be slow velocity of vertical saccades, a tendency to fall on the pull-test within 3 years, Parkinsonism, akinetic-rigid, predominantly axial, and levodopa resistant and frontal cognitive/behavioral presentation. Also significant, but somewhat less correlated (“Level 3” clinical features) can be frequent macro square wave jerks or “eyelid opening apraxia”; more than two steps backward on the pull-test within 3 years, Parkinsonism, with tremor and/or asymmetric and/or levodopa responsive, and corticobasal syndrome. Patients treatable with compositions described herein can have at least one Level 1 or Level 2 clinical feature. Many patients can have a combination of clinical features drawn from the Level 1, Level 2 and Level 3 clinical features. Various types of corticobasal degeneration patients can be treated with compositions according to the disclosure. Patients with probable corticobasal syndrome and/or probable corticobasal degeneration can be treated.

Argyrophilic grain disease (AGD) can present without any unique clinical features. AGD can manifest as AD and so AGD can be considered to be clinically the same as AD, but with evidence that the patient lacks amyloid pathology. Amyloid pathology can be discounted by examining CSF levels of beta-amyloid 42 and/or using beta-amyloid PET imaging. AGD can lack acetylated tau in inclusions compared to other 4R tauopathies. Like AGD, globular glial tauopathy (GGT) can lack a defining clinical syndrome, presenting with a combination of frontotemporal dementia, motor neuron disease and/or extrapyramidal features. Identification of GGT patients can involve eliminating other pathologies using imaging, biomarkers and differential diagnosis. Wandering in male or female human patients suspected of having dementia resulting from a stroke can be treated with compositions described herein. Dementia related to a cerebrovascular accident (CVA, or stroke) can be referred to as vascular dementia.

Wandering or other symptoms in male or female human patients suspected of having dementia from a traumatic brain injury (TBI) can be treated with compositions described herein. TBI is an environmental risk factor for dementia. There can also be a genetic component affecting dementia resulting from TBI. A history of TBI and inheritance of an APOE 84 allele can be associated with a 10-fold increased risk of dementia, while APOE 84 in the absence of TBI can result in only a 2-fold increased risk. Autopsy studies have shown that β-amyloid plaques and neurofibrillary tangles (NFTs) were present in up to one-third of patients with prolonged survival after a single TBI. Patients experiencing multiple TBIs, such as professional athletes who sustained multiple concussions), can experience dementia related to chronic traumatic encephalopathy (CTE), previously known as dementia pugilistica, or boxer's dementia. CTE autopsies have shown global atrophy of the brain, with thinning of the corpus callosum and enlarged ventricles and cavum septum pellucidum.

Compositions of the present disclosure can be used to treat wandering or other symptoms in patients diagnosed with amyotrophic lateral sclerosis (ALS) and other motor neuron diseases. Other motor neuron diseases comprise, for example, X-linked spinobulbar muscular atrophy (Kennedy's disease), adult Tay-Sach's disease, spinal muscular atrophy, multifocal motor neuropathy with conduction block, primary lateral sclerosis, and familial spastic paraplegia. Upper and lower neuron degeneration can be manifest. Symptoms of ALS and other motor neuron diseases can depend on whether corticospinal neurons, lower motor neurons, or both are affected and the degree of degeneration. Symptoms can include, for example, muscle twitches in the arm, leg, shoulder, or tongue; muscle cramps; tight and stiff muscles (spasticity); muscle weakness affecting an arm, a leg, the neck, or diaphragm; slurred and nasal speech; or difficulty chewing or swallowing; or any combination thereof. Composition of the present disclosure can further comprise or be administered together with one or more additional APIs, for example, riluzole, edaravone, or both to a patient diagnosed with ALS or another motor neuron disease.

Methods of manufacturing the compositions of the present disclosure are also encompassed by this disclosure. Any suitable manufacturing process can be used to manufacture the compositions. A composition can be manufactured as a concentrate and then diluted before administration. A dry or liquid concentrate can be manufactured. A freeze dry concentrate can be manufactured and then reconstituted for administration. A composition can be manufactured in separate admixture for mixture prior to administration to a patient. Use of a rho kinase inhibitor to manufacture a composition is also provided by the present disclosure, for example, to treat one or more neurodegenerative diseases. Use of a rho kinase inhibitor in a composition of the present disclosure to treat one or more neurodegenerative disease is further provided by the present disclosure.

A method of forming fasudil-comprising liposomes is provided. The fasudil-comprising liposomes can be formulated for oral administration. The method can comprise one or more of the following steps. A diacylglyceride, cholesterol, a rho kinase inhibitor, and, optionally, a cellulose ether can be mixed in a volatile organic solvent to produce a first composition. The diacylglyceride can be pegylated. A combination of pegylated and unmodified diacylglyceride can be used. The rho kinase inhibitor can comprise fasudil, a pharmaceutically acceptable salt thereof, a hydrate thereof, a prodrug thereof, a substituted derivative thereof, or a metabolite thereof, or any combination thereof. The first mixture can be subjected to evaporation to produce a second composition. An aqueous solution can be applied to the second composition to form a third composition. Energy can be imparted to the third composition to produce a fourth composition, the fourth composition comprising the fasudil-comprising liposomes. Any suitable volatile organic solvent can be employed. For example, the volatile organic solvent can comprise chloroform, or methanol, or both. The evaporation can be performed, for example, below ambient pressure, or above ambient temperature, or both. The aqueous solution can comprise an acidic solution, a neutral solution, or an alkaline solution. The energy can be imparted using any suitable technique. For example, the energy can be imparted using sonication. A method of forming fasudil-containing enteric coated liposomes is provided. The method can comprise, for example, producing liposomes comprising fasudil, a pharmaceutically acceptable salt thereof, a hydrate thereof, a prodrug thereof, a substituted derivative thereof, or a metabolite thereof, or any combination thereof; and coating the fasudil-comprising liposomes to form the fasudil-containing enteric coated liposomes.

Production of liposomes can involve any suitable technique or combination of techniques. A technique or a combination of techniques for liposomal formation, processing, and characterization can be used as described, for example, in Lombardo et al., “Methods of Liposomes Preparation: Formation and Control Factors of Versatile Nanocarriers for Biomedical and Nanomedicine Application,” Pharmaceutics, 14, 543, 1-49 (2022), which is incorporated by reference in its entirety. For example, liposomes can be produced through one or more of the following stages. Lipids can be dissolved in an organic solvent to form a lipidic solution. The lipidic solution can be dried down from the organic solvent. The lipid of the lipidic solution can be hydrated with an aqueous medium, before or after the dry down, followed by agitation or stirring for an initial formation of liposomes. Downsizing, or alteration of lamellarity, or both of the initially formed liposomes can be performed. Post-formation processing, for example, purification, or sterilization, or both of the liposomes can be performed. The liposomes so produced can then be characterized. Characterization can also be performed during or between the various stages of liposomal production.

A thin-film hydration (TFH) method, also known as the Bangham method, can be employed to form liposomes. Phospholipid components can be dissolved in an organic solvent, for example, dichloromethane, chloroform, ethanol, or methanol, or any combination thereof. The organic solvent can be removed under vacuum at elevated temperature, for example, from about 45° C. to about 60° C., to remove the organic solvent. Evaporation can be performed, for example, using a stream of an inert gas such as nitrogen or argon or by rotary evaporation. A homogeneous, dry, thin-lipid film can result from the evaporation and can comprise a stack of lipid bilayers. An aqueous medium, which can be buffered, is used to hydrate the lipid film. Hydration can be performed at an elevated temperature, for example, from about 60° C. to about 70° C. over a period of time, for example, from about 1.0 hour to about 2.0 hours. The temperature of hydration can be set at a temperature above the phase-transition temperatures of the lipid components. Agitation such as stirring can be performed during hydration to aid in liposome formation. A prolonged, for example, overnight, incubation period at a reduced temperature, for example, at about 4° C., is then performed to advance lipid hydration and formation of the liposomes. The resulting composition can have elevated in multi-lamellar vesicles (MLVs) with varying degrees of heterogeneity and lamellarity. Such MLVs can be downsized using, for example, extrusion, sonication, or homogenization, or any combination thereof. Once downsized, the liposomes can be purified, for example, by filtration, centrifugation, chromatography, or dialysis, or any combination thereof. Fasudil can be loaded at any desired stage, for example, during an organic solvent stage, a hydration stage, a laminar stage, a multi-lamellar stage, or a post liposomal formation stage, or any combination thereof. Fasudil, or another compound, or both, can be loaded into liposomes using any suitable technique. Drug loading can comprise passive loading, or active loading, or both.

Alternatives to the TFH method can include, for example, one or more of the following. A detergent removal or depletion method can be employed to form liposomes. A solvent injection method can be employed to form liposomes, for example, using ethanol or ether. Liposomes can be formed using a reverse-phase evaporation method. Still other methods for liposomal formation include, freeze-drying (lyophilization), supercritical fluid-assisted methods, a microfluidic (channel) method, and a membrane contractor method. Supercritical fluid-assisted methods can include, for example, a supercritical reverse-phase evaporation (SC-RPE) method, a supercritical anti-solvent (SAS) method, a rapid expansion of supercritical solution (RESS) method, a supercritical-assisted liposome formation (SuperLip) method, and a depressurization of an expanded liquid organic solution into aqueous suspension (DELOS) method.

Once formed, the liposomes can be downsized and be subjected to various post-formation processing. The liposomes, for example, in the form of multi-lamellar vesicles (MLVs), can be subject to sonication, extrusion, and homogenization. Examples of sonication include bath sonication and probe sonication. Extrusion can comprise passing a liposomal composition through one or more pore-containing membranes, for example, with sizes of from about 25 nm to about 1.0 mm. Homogenization of a liposomal composition can comprise a high-pressure homogenization process. High-pressure homogenization can comprise forcing the liposomal composition through an orifice at pressure against a target. Liposomes can be modified in any desired way. For example, liposomes can be coated. Modifications can help control targeting, or stability, or both of the liposomes. Liposomes can be modified with one or more targeting moieties, for example, a ligand, recognizable by a corresponding receptor on a cell surface. Binding of the ligand to its receptor can assist in concentrating liposomes at a particular target and absorption into target cell types. Targeting moieties can comprise a polypeptide, a carbohydrate, a lipid, a polynucleotide, a metal, a synthetic polymer, or a small molecule, or any combination thereof.

Before or after drug loading, or before or after downsizing, or both, liposomes can be characterized by one or more techniques, for example, calorimetry, microscopy, light scattering, zeta-potential, X-ray scattering, neutron scattering, or spectroscopy, or any combination thereof. Examples of scattering techniques can include a small-angle X-ray (SAXS) technique, a small-angle neutron scattering (SANS) technique, and a dynamic light scattering technique. Diffraction techniques can comprise, for example, X-ray diffraction. Microscopy techniques can comprise, for example, electron microscopy, scanning electron microscopy (SEM), transmission electron microscopy (TEM), scanning tunneling electron microscopy (STM), atomic force microscopy (AFM), light microscopy, fluorescence microscopy, and confocal microscopy. Spectroscopy techniques can comprise UV spectroscopy, FT-IR spectroscopy, electron paramagnetic resonance (EPR), electron spin resonance (ESR), and circular dichroism. calorimetry can comprise, for example, differential scanning calorimetry (DSC) and isothermal titration calorimetry (ITC). Nuclear magnetic resonance (NMR) can also be used to characterize liposomes.

Polydispersity Index (PDI) refers to the non-uniformity of particle size distribution (liposomal size distribution), which can be measured, for example, through dynamic light scattering. The PDI of liposomal compositions can be less than about 1.0, less than about 0.3, from about 0.001 to about 1.0, from about 0.01 to about 0.750, from about 0.1 to about 0.6, or from about 0.2 to about 0.5, or more than about 1.0, or any intervening value, or any range therebetween.

Zeta potential is an example of a parameter by which liposomes can be characterized. Zeta potential can affect colloidal stability, drug loading, pharmacokinetics, biodistribution, and cellular affinity of liposomes. The zeta potential relates to electrostatic effects of liposomes and is influenced by the lipid components of the liposomes including, for example, the head groups of lipids. A mean zeta value of the plurality of liposomes can be from about −50 mv to about −5 mv, from about −45 mv to about −10 mv, from about −40 mv to about −15 mv, from about −30 mv to about −20 mv, or any intervening zeta value, or any range of zeta values therebetween. Liposomes can have neutral zeta values. Liposomes can also have positive zeta values, for example from about +1.0 mv to about +50 mv, for example, positive counterpart zeta values and range to the negative zeta values and ranges described. High zeta values can provide electrical stability of liposomes. Low zeta potentials can favor aggregation, or flocculation, or both of liposomes. Zeta potential can be measured using any suitable technique, for example, by measuring the electrophoretic mobility μE using principles of phase analysis light scattering (PALS). A Zetasizer device available from Malvern Panalytical (Spectris), or equivalent device, can be used to measure zeta potential and obtain zeta values.

### EXAMPLES

Fasudil-containing liposomes were generated using various procedures as set forth in the following examples. A formulation number “F #” is assigned to each formulation. A parenthetical indicates that a particular formulation was a repetition of the formulation number indicated in the parenthetical. Results for selected formulations are set forth in Tables 2 and 3. Polydispersity Index (PDI) and zeta potential were measured.

TABLE 2

| | | | | Drug | | | Zeta |
|---|---|---|---|---|---|---|---|
| | | | % | loading | Size | | potential |
| F# | Formulations | Drug added | Entrapment | mg/mL | (nm) | PDI | mv |
| F1 | DPPC:Cholesterol(7:3) | 15 mg drug | 0.3 | 0.1 | 156 | 0.092 | −20.1 |
| | (10 mM) & | dissolved in | | | | | |
| | Methanol:chloroform (2:1) | 2 ml water | | | | | |
| F2 | DPPC:Cholesterol(5:5) | 15 mg drug | 0.7 | 0.1 | 172 | 0.1 | −16.3 |
| | (10 mM) & | dissolved in | | | | | |
| | Methanol:chloroform (1:1) | 0.5 ml of water | | | | | |
| F3 | DSPE:DPPC:Cholesterol | 15 mg drug | 0.7 | 0.1 | 213 | 0.112 | −18.4 |
| | (1:1:1) (20 mM), | dissolved in | | | | | |
| | Methanol:chloroform (2:1) | 0.5 ml of water | | | | | |
| F4 | DSPE:DPPC:Cholesterol | 15 mg drug | 0.9 | 0.1 | 163.1 | 0.123 | −20.1 |
| | (3:4:3 (20 mM), | dissolved in | | | | | |
| | Methanol:chloroform (2:1) | 0.5 ml of water | | | | | |
| F5 | DSPE:DPPC:Cholesterol | 15 mg drug | 0.9 | 0.0 | 209.3 | 0.21 | −20.3 |
| | (1:1:1) (20 mM), | dissolved in | | | | | |
| | Methanol:chloroform (2:1) | 0.5 ml of water | | | | | |
| F6 | DSPE:DPPC:Cholesterol | 15 mg drug | 3.9 | 0.0 | 186.2 | 0.15 | −21.0 |
| | (3:4:3 (20 mM), | dissolved in | | | | | |
| | Methanol:chloroform (2:1) | 0.5 ml of water | | | | | |
| F7 | DPPC:Cholesterol | 15 mg drug | 18.6 | 2.4 | 160 | 0.09 | −18.2 |
| | (1:1) (20 mM) | dissolved with | | | | | |
| | Methanol:chloroform (2:1) | lipids | | | | | |
| F8 | DPPC:Cholesterol | 15 mg drug | 0.4 | 0.0 | 225 | 0.212 | −15.4 |
| | (1:1) (30 mM) | dissolved with | | | | | |
| | Methanol:chloroform (2:1) | lipids | | | | | |
| F9 | DPPC:Cholesterol | 15 mg drug | 3.9 | 2.8 | 198.1 | 0.11 | −22.3 |
| | (6:4) (30 mM) | dissolved with | | | | | |
| | Methanol:chloroform (2:1) | lipids | | | | | |
| F10 | DPPC:Cholesterol | 15 mg drug | 25.5 | 0.4 | 186.2 | 0.121 | −21.5 |
| | (1:1) (30 mM) | dissolved with | | | | | |
| | Methanol:chloroform (2:1) | lipids | | | | | |
| F11 | DSPE:DPPC:Cholesterol | 15 mg drug | 1.2 | 0.2 | 201.3 | 0.092 | −19.2 |
| | (3:4:3 (20 mM), | dissolved with | | | | | |
| | Methanol:chloroform (2:1) | lipids | | | | | |
| F12 | DSPE:DPPC:Cholesterol | 15 mg drug | 0.5 | 0.1 | 192 | 0.218 | −20.4 |
| | (1:1:1) (20 mM), | dissolved with | | | | | |
| | Methanol:chloroform (2:1) | lipids | | | | | |
| F13 | DSPE:DPPC:Cholesterol | 15 mg drug | 2.9 | 0.3 | 313 | 0.224 | −22 |
| | (3:4:3 (30 mM), | dissolved with | | | | | |
| | Methanol:chloroform (2:1) | lipids | | | | | |
| F14 | DSPE:DPPC:Cholesterol | 15 mg drug | 0.5 | 0.1 | 254.2 | 0.114 | −21.9 |
| | (1:1:1) (30 mM), | dissolved with | | | | | |
| | Methanol:chloroform (2:1) | lipids | | | | | |
| F15 | DSPE:DPPC:Cholesterol | 15 mg drug | 0.7 | 0.1 | 204.2 | 0.1 | −17.6 |
| | (3:4:3 (30 mM), | dissolved in | | | | | |
| | Methanol:chloroform (2:1) | 0.5 ml of water | | | | | |
| F16 | DPPC:Cholesterol | 15 mg drug | 7.0 | 0.6 | 228 | 1 | −21.4 |
| | (1:1) (30 mM), | was dissolved | | | | | |
| | Methanol:chloroform (2:1) | in water | | | | | |
| F17 | DPPC:Cholesterol | 15 mg drug | 26.5 | 2.2 | 198 | 0.12 | −20 |
| | (1:1) (40 mM), | dissolved with | | | | | |
| | Methanol:chloroform (2:1) | lipids | | | | | |
| F18 | DPPC:Cholesterol | 15 mg drug | 62.9 | 5.2 | 238 | 0.21 | −22.9 |
| | (1:1) (40 mM), | dissolved with | | | | | |
| | Methanol:chloroform (2:1) | lipids | | | | | |
| F19 | DPPC:Cholesterol | 15 mg drug | 24.0 | 2.2 | 193.7 | 0.09 | −21 |
| | (1:1) (30 mM), | dissolved with | | | | | |
| | Methanol:chloroform (2:1) | lipids | | | | | |
| F20 | DPPC:Cholesterol | 15 mg drug | 60.6 | 5.7 | 209.2 | 0.11 | −21.3 |
| | (1:1) (30 mM), | dissolved with | | | | | |
| | Methanol:chloroform (2:1) | lipids | | | | | |
| F21 | DPPC:Cholesterol | 15 mg drug | 54.3 | 5.1 | 212 | 0.098 | −19.4 |
| (F20) | (1:1) (30 mM), | dissolved with | | | | | |
| | Methanol:chloroform (2:1) | lipids | | | | | |
| F23 | DPPC:Cholesterol | 15 mg drug | 13.8 | 1.1 | 250 | 0.312 | −20.3 |
| | (1:1) (40 mM), | dissolved with | | | | | |
| | Methanol:chloroform (2:1) | lipids | | | | | |
| F24 | DPPC:Cholesterol | 30 mg drug | 62.9 | 10.8 | 240 | 0.156 | −23.1 |
| | (1:1) (30 mM), | dissolved with | | | | | |
| | Methanol:chloroform (2:1) | lipids | | | | | |
| F25 | DPPC:Cholesterol | 30 mg drug | 72.1 | 11.1 | 246 | 0.23 | −24.5 |
| | (1:1) (40 mM), | dissolved with | | | | | |
| | Methanol:chloroform (2:1) | lipids | | | | | |
| F26 | DPPC:Cholesterol | 30 mg drug | 60.8 | 10.4 | 248 | 0.312 | −25.6 |
| (F24) | (1:1) (30 mM), | dissolved with | | | | | |
| | Methanol:chloroform (2:1) | lipids | | | | | |
| F27 | DPPC:Cholesterol | 30 mg drug | 68.5 | 10.5 | 253 | 0.43 | −26 |
| (F25) | (1:1) (40 mM), | dissolved with | | | | | |
| | Methanol:chloroform (2:1) | lipids | | | | | |
| F28 | DPPC:Cholesterol | 30 mg drug | 66.4 | 11.4 | 232.1 | 0.102 | −21.4 |
| (F24) | (1:1) (30 mM), | dissolved with | | | | | |
| | Methanol:chloroform (2:1) | lipids | | | | | |
| F29 | DPPC:Cholesterol | 30 mg drug | 63.0 | 10.8 | 212 | 0.2 | −20.6 |
| (F24) | (1:1) (30 mM), | dissolved with | | | | | |
| | Methanol:chloroform (2:1) | lipids | | | | | |
| F30 | DPPC:Cholesterol | 30 mg drug | 65.2 | 11.2 | 210 | 0.212 | −22.7 |
| (F24) | (1:1) (30 mM), | dissolved with | | | | | |
| | Methanol:chloroform (2:1) | lipids | | | | | |
| F31 | DPPC:Cholesterol | 30 mg drug | 70.5 | 10.9 | 247 | 0.1 | −26 |
| (F25) | (1:1) (40 mM), | dissolved with | | | | | |
| | Methanol:chloroform (2:1) | lipids | | | | | |
| F32 | DPPC:Cholesterol | 30 mg drug | 71.6 | 11.0 | 228.1 | 0.156 | −23.1 |
| (F25) | (1:1) (40 mM), | dissolved with | | | | | |
| | Methanol:chloroform (2:1) | lipids | | | | | |
| F33 | DPPC:Cholesterol | 30 mg drug | 69.9 | 10.8 | 226 | 0.112 | −24.3 |
| (F25) | (1:1) (40 mM), | dissolved with | | | | | |
| | Methanol:chloroform (2:1) | lipids | | | | | |
| F34 | DPPC:Cholesterol | 15 mg drug | 19.5 | 1.7 | 428 | 1 | −21.4 |
| | (1:1) (30 mM), | was dissolved | | | | | |
| | Methanol:chloroform (2:1) | in water | | | | | |
| F35 | DPPC:Cholesterol | 15 mg drug | 19.8 | 1.7 | 298 | 0.21 | −18.7 |
| | (1:1) (30 mM), | was dissolved | | | | | |
| | Methanol:chloroform (2:1) | in 0.1N | | | | | |
| | | NaOH | | | | | |
| F36 | DPPC:Cholesterol | 15 mg drug | 18.1 | 1.5 | 256 | 0.354 | −22.1 |
| | (1:1) (30 mM), | was dissolved | | | | | |
| | Ethanol and water | in NAOH | | | | | |
| F37 | DPPC:Cholesterol | 15 mg drug | 3.7 | 0.3 | 163 | 0.05 | −25.6 |
| | (6:4) (30 mM), | was dissolved | | | | | |
| | Diethyl ether 10 ml | in Diethyl | | | | | |
| | | ether along | | | | | |
| | | with lipids | | | | | |
| F38 | DPPC:Cholesterol | 15 mg drug | 0.7 | 0.1 | 160 | 0.048 | −24 |
| | (6:4) (30 mM), | was dissolved | | | | | |
| | Diethyl ether 10 ml | in 20 ml of | | | | | |
| | | water | | | | | |
| F39 | DPPC:Cholesterol | 15 mg drug | 10.4 | 0.9 | 172.7 | 0.09 | −24.3 |
| | (6:4) (30 mM), | was dissolved | | | | | |
| | Diethyl ether 10 ml | in 20 ml of | | | | | |
| | | 0.1N NAOH | | | | | |
| F40 | DPPC:Cholesterol | 15 mg drug | 5.2 | 0.4 | 206 | 0.19 | −29 |
| | (1:1) (30 mM), | was dissolved | | | | | |
| | Methanol:chloroform (2:1) | in 0.5 ml of | | | | | |
| | | 0.1N NaOH | | | | | |
| F41 | DPPC:Cholesterol | 15 mg drug | 0.5 | 0.0 | 475 | 0.234 | −26.7 |
| | (6:4) (30 mM), 10 | was dissolved | | | | | |
| | mL of Diethyl ether | in 2 ml of water | | | | | |
| F42 | DPPC:Cholesterol | 15 mg drug | 4.3 | 0.4 | 298.1 | 0.106 | −21.6 |
| | (6:4) (30 mM), | dissolved with | | | | | |
| | Dichloromethane | lipids | | | | | |
| F43 | DPPC:Cholesterol | 15 mg drug | 10.3 | 0.9 | 201 | 0.212 | −25.3 |
| | (6:4) (30 mM), | dissolved in | | | | | |
| | Dichloromethane | 0.5 ml of water | | | | | |
| F44 | DPPC:Cholesterol | 15 mg drug | 58.0 | 5.0 | 208.4 | 0.158 | −22.0 |
| | (6:4) (30 mM), | dissolved with | | | | | |
| | Methanol:chloroform (2:1) | lipids | | | | | |
| F45 | DPPC:Cholesterol | 15 mg drug | 81.9 | 7.0 | 226.1 | 0.16 | −23.1 |
| | (1:1) (30 mM), | dissolved with | | | | | |
| | Methanol:chloroform (2:1) | lipids | | | | | |
| F46 | DPPC:Cholesterol | 30 mg drug | 26.9 | 4.6 | 256 | 0.43 | −21.0 |
| | (6:4) (40 mM), | dissolved with | | | | | |
| | Methanol:chloroform (2:1) | lipids | | | | | |
| F47 | DPPC:Cholesterol | 30 mg drug | 85.1 | 13.1 | 235 | 0.11 | −20.5 |
| | (1:1) (40 mM), | dissolved with | | | | | |
| | Methanol:chloroform (2:1) | lipids | | | | | |
| F58 | DPPC:Cholesterol (1:1) | 30 mg drug | 86.1 | 9.2 | 296.5 | 0.62 | −26.8 |
| | (40 mM), 100 mg HPMC, | dissolved with | | | | | |
| | Methanol:chloroform (2:1) | lipids | | | | | |
| F62 | DPPC:Cholesterol | 30 mg drug | 84.41437929 | 9.0442857 | 329.2 | 0.45 | −31.5 |
| | (1:1) (40 mM) and | dissolved with | | | | | |
| | HPMC (100 mg) | lipids | | | | | |
| | Methanol:chloroform (2:1) | | | | | | |
| F68 | DPPC:Cholesterol(1:1) | 30 mg drug | 66.06 | 10.163 | 352.1 | 0.48 | −25.1 |
| | (40 mM), DSPE-PEG | dissolved with | | | | | |
| | 2000 (0.5 mM), | lipids | | | | | |
| | Methanol:chloroform (2:1) | | | | | | |
| F69 | DPPC:Cholesterol (1:1) | 30 mg drug | 85.09 | 8.509 | 289.2 | 0.167 | −23.1 |
| | and DSPE-PEG 2000 | dissolved with | | | | | |
| | (0.5 mM) HPMC | lipids | | | | | |
| | (100 mg) (40 mM), | | | | | | |
| | Methanol:chloroform (2:1) | | | | | | |

Table 3 shows attempts to increase drug loading by increasing the drug: lipid ratio with the formulations listed. Drug load appeared to plateau around 20 mg/mL.

TABLE 3

| | Lipids (mM) | | | | |
|---|---|---|---|---|---|
| | (DPPC:CHOL 1:1) | | Drug | | Formu- |
| | in 10 mL | Drug | loaded | % | lation |
| F# | MtOH:Chloroform (2:1) | (mg) | (mg/mL) | Loading | Code |
| F40 | 30 | 15 | 0.783 | 0.45 | FAS-Lip-1 |
| F45 | 30 | 15 | 12.3 | 7.02 | FAS-Lip-2 |
| F47 | 40 | 30 | 25.5 | 13.1 | FAS-Lip-3 |
| F48 | 30 | 15 | 11.9 | 6.8 | FAS-Lip-2 |
| (F45) | | | | | |
| F49 | 40 | 30 | 26.6 | 13.6 | FAS-Lip-3 |
| (F47) | | | | | |
| F50 | 40 | 40 | 22.2 | 11.4 | FAS-Lip-4 |
| F51 | 40 | 50 | 21.2 | 10.8 | FAS-Lip-5 |
| F52 | 40 | 60 | 26.8 | 13.8 | FAS-Lip-6 |
| F53 | 40 | 40 | 22.2 | 11.4 | FAS-Lip-4 |
| (F50) | | | | | |
| F54 | 40 | 50 | 24.8 | 12.7 | FAS-Lip-5 |
| (F51) | | | | | |
| F55 | 40 | 40 | 22.4 | 11.5 | FAS-Lip-4 |
| (F50) | | | | | |
| F56 | 40 | 50 | 21.4 | 11 | FAS-Lip-5 |
| (F51) | | | | | |
| F60 | 40 | 60 | 20.4 | 10.5 | FAS-Lip-6 |
| (F52) | | | | | |
| F61 | 40 | 100 | 19.8 | 7.3 | FAS-Lip-7 |
| F63 | 40 | 100 | 19.1 | 7.1 | FAS-Lip-7 |
| F64 | 60 | 100 | 20.4 | 14.2 | FAS-Lip-8 |
| F65 | 80 | 100 | 18.8 | 17.4 | FAS-Lip-9 |

Averages from Table 3 are shown in Table 4 (SD=standard deviation).

TABLE 4

| | Drug Load | | % | | Size | |
|---|---|---|---|---|---|---|
| | (mg) | | Loading | | (nm) | |
| F Code | Average | SD | Average | SD | Average | SD |
| FAS-Lip-1 | 0.783 | | 0.45 | | 206 | |
| FAS-Lip-2 | 12.1 | 0.3 | 6.9 | 0.2 | 237.0 | 15.6 |
| FAS-Lip-3 | 26.1 | 0.8 | 13.4 | 0.4 | 264.5 | 41.7 |
| FAS-Lip-4 | 22.3 | 0.1 | 11.4 | 0.1 | 233.4 | 7.3 |
| FAS-Lip-5 | 22.5 | 2.0 | 11.5 | 1.0 | 240.7 | 9.9 |
| FAS-Lip-6 | 23.6 | 4.5 | 12.2 | 2.3 | 261.0 | 18.4 |
| FAS-Lip-7 | 19.8 | | 7.3 | | | |

Coating data is shown in Table 5. For coating of Fasudil liposomes, the following procedure was followed:
1. Eudragit L-100 or S-100 was dissolved in 5 ml of ethanol. This polymer solution was stirred until a clear solution was formed.
2. Once the clear solution was obtained, fabricated liposomes were added to it and were stirred room temperature at 200 rpm for 3 hours (no temperature).
3. After 3 hours, the solution was centrifuged at 15000 rpm for 15 minutes at 4° C. by addition of excess water and a pellet was obtained.
4. The obtained packet was suspended in 1 ml of water and subjected to freeze-thaw (3 cycles, 3 minutes for each cycle).
5. The formulations were lysed using ACN: Water (50:50), dichloromethane (methylene chloride; DCM), and ethanol.
For coating with HPMC, a similar procedure was followed except it was dissolved in ethanol: DCM (2:1) 5 ml. The surface of liposome was also modified by incorporating HPMC and/or pegylated-DSPE into the liposomes. HPMC and/or pegylated-DSPE was dissolved along with lipids and fasudil in organic solvent (see, e.g., Example 21).

TABLE 5

| | | | Drug | | | |
|---|---|---|---|---|---|---|
| | Lipids | Drug | loaded | % | Formulation | |
| Formulation | (mM) | (mg) | (mg) | Loading | Code | Comments |
| F47-L100 | 40 | 30 | 20.9 | 10.7 | FAS-Lip-3L | Coated with Eudragit L100 |
| F47-S100 | 40 | 30 | 17.3 | 8.9 | FAS-Lip-3S | Coated with Eudragit S100 |
| F47-HPMC | 40 | 30 | 19.6 | 10.1 | FAS-Lip-3H | Coated with HPMC |
| F58 | 40 | 30 | 25.8 | 9.2 | FAS-Lip-3HD | 100 mg HPMC dissolved in lipid film |
| F59 | 40 | 30 | 24.9 | 8.9 | FAS-Lip-3HD | 100 mg HPMC dissolved in lipid film |
| F47-HPMC | 40 | 30 | 18.4 | 9.4 | FAS-Lip-3H | Coated with HPMC |
| F47-L100 | 40 | 30 | 20.8 | 10.7 | FAS-Lip-3L | Coated with Eudragit L100 |
| F47-S100 | 40 | 30 | 17.5 | 9 | FAS-Lip-3S | Coated with Eudragit S100 |
| F47-HPMC | 40 | 30 | 18 | 9.22 | FAS-Lip-3H | Coated with HPMC |
| F62 | 40 | 30 | 25.3 | 9.04 | FAS-Lip-3HD | 100 mg HPMC dissolved in lipid film |

Averages from Table 5 are shown in Table 6 (SD=standard deviation).

TABLE 6

| | Drug Load | | % | | Size | |
|---|---|---|---|---|---|---|
| Formulation | (mg) | | Loading | | (nm) | |
| Code | Average | SD | Average | SD | Average | SD |
| FAS-Lip-3 | 26.1 | 0.8 | 13.4 | 0.4 | 264.5 | 41.7 |
| FAS-Lip-3L | 20.9 | 0.1 | 10.7 | 0.0 | 275.0 | |
| FAS-Lip-3S | 17.4 | 0.1 | 9.0 | 0.1 | 291.2 | |
| FAS-Lip-3H | 19.0 | 0.8 | 9.8 | 0.5 | 296.5 | 7.8 |
| FAS-Lip-3HD | 25.4 | 0.6 | 9.1 | 0.2 | 303.4 | 9.7 |

### Example 1

For formulations F1 and F2, lipids were dissolved in organic solvent, rotary evaporated for about 45 minutes to form a thin film. The film was hydrated with fasudil aqueous solution (15 mg/2 ml). The resulting solution was sonicated for 5 minutes, and probe sonicated for 6 minutes (on for 10 second-off for 10 second cycles). The solution was then centrifuged at 15K for 15 minutes. Percent entrapment was 0.3%-0.7%.

### Example 2

For formulations F3-F6, ammonium sulfate gradient loading was employed. Lipids were dissolved in organic solvent and rotary evaporated to form a film. The thin film was hydrated with ammonium sulfate solution, and sonicated. Column separation was performed to remove ammonium sulfate. Fasudil was dissolved in water, and mixed with the liposomes, heated to 65° C. to load the drug into the liposomes. Centrifugation was performed to remove free drug. The percent encapsulation was 0.7%-3.9%.

### Example 3

For formulations F7-F9 and F11-F14, lipids and drug were dissolved in the organic solvents, and then rotary evaporated to form a film. The film was hydrated with 2 mL of 0.5% chitosan solution in HCl, and sonicated. The resulting liposomal solution was centrifuged to remove free drug.

### Example 4

For formulations F10, F16-F18, and F21 were prepared the same as formulations F7-F9. Further, after centrifugation, the formulation was freeze thawed for 3 cycles (one cycle=liquid nitrogen for 3 minutes, then water bath at 45° C. for 3 minutes).

### Example 5

Formulation F15 was prepared the same as formulations F3-F6. Further, after centrifugation, the formulation was freeze thawed for 3 cycles (one cycle=liquid nitrogen for 3 minutes, then placed in a water bath at 45° C. for 3 minutes).

### Example 6

For formulations F19, F20, and F22-F33, fasudil and lipids were dissolved in organic solvents, and then rotary evaporated to form a film. The resulting film was hydrated with water, sonicated, and centrifuged. Three freeze thaw cycles were then performed.

### Example 7

For formulation F34, lipids were dissolved in the organic solvents to form a lipid solution, and 1 mL of fasudil aqueous solution (15 mg/mL) was mixed with the lipid solution. The resulting solution was probe sonicated to form an emulsion. The emulsion was rotary evaporated to remove organic solvent. The liposomes produced were then bath sonicated and washed.

### Example 8

For formulation F35, lipids were dissolved in the organic solvents, to form a lipid solution. 1 mL of 0.1N NaOH containing 15 mg fasudil was mixed with the lipid solution. The resulting solution was probe sonicated to form an emulsion. The emulsion was rotary evaporated to remove organic solvent. The liposomes were bath sonicated and washed.

### Example 9

For formulation F36, lipids were dissolved in EtOH/Water (1:1) at 60° C., 1 mL of NaOH containing 15 mg FAS was added under stirring. The resulting solution was rotary evaporated to remove organic solvent and the resulting film was hydrated with water. For liposomal formation, 1N NaOH was used, and were bath sonicated and washed.

### Example 10

For formulation F37, lipids were dissolved in 10 mL diethyl ether, and 15 mg of fasudil was added. The organic phase along with the fasudil was added to 20 mL of water, and stirred at 60° C. for an hour. Diethyl ether was removed by rotary evaporation. The liposomes were water bath sonicated and washed.

### Example 11

For formulation F38, lipids were dissolved in 10 mL diethyl ether then added to 20 mL of fasudil aqueous solution (containing 15 mg of fasudil). The resulting solution was stirred at 60° C. for one hour. Diethyl ether was removed by rotary evaporation under reduced pressure. The resulting liposomes were water bath sonicated and washed.

### Example 12

For formulation F39, lipids were dissolved in diethyl ether and added to 20 mL 0.1 N NaOH solution containing 15 mg of fasudil. The resulting solution was stirred at 60° C. for an hour. Diethyl ether was removed using stirring. The resulting liposomes were water bath sonicated and washed.

### Example 13

For formulation F40, lipids were dissolved in 10 ml of organic solvent (methanol:chloroform (2:1)). The resulting solution was rotary evaporated for about 45 minutes to form a film. The film was hydrated with 2 mL 0.1 N NaOH containing 15 mg of fasudil. The hydrated film was water bath sonicated for 5 minutes and probe sonicated for 6 minutes. Centrifugation was performed to remove free fasudil.

### Example 14

For formulation F41, lipids were dissolved in 10 mL of diethyl ether. 1 mL water containing 15 mg of fasudil was added to the oil phase. The resulting solution was probe sonicated for 6 minutes. Rotary evaporation was performed to remove diethyl ether. The liposomes were bath sonicated and washed.

### Example 15

For formulation F42, 30 mM lipids (DPPC: CHOL (3:2)) and 15 mg of fasudil were dissolved in 10 mL dichloromethane and rotary evaporated for 45 minutes to form a film. The film was hydrated with 2 mL of 1×PBS. The hydrate film was bath sonicated for 5 minutes and probe sonicated for 6 minutes. The resulting liposomal composition was centrifuged at 15K rpm for 15 minutes.

### Example 16

For formulation F43, 30 mM lipids (DPPC: CHOL (3:2)) were dissolved in 10 mL dichloromethane, 15 mg of fasudil was dissolved in 0.5 mL water, and the two solutions were mixed. The resulting solution was rotary evaporated for 45 minutes to form a film. Liposomes were bath sonicated for 5 minutes and probe sonicated for 6 minutes. Centrifugation at 15K rpm for 15 minutes was performed.

### Example 17

For formulation F44, 30 mM lipids (DPPC: CHOL (3:2)) and 15 mg of fasudil were dissolved in 10 mL organic solvent (Methanol:chloroform (2:1)). Rotary evaporation of the resulting mixture was performed for 45 minutes to form a film. The film was hydrated with 2 mL of 1×PBS (pH 7). The hydrated film was bath sonicated for 5 minutes and probe sonicated for 6 minutes. The resulting liposomal composition was then centrifuged.

### Example 18

For formulation F45, 30 mM lipids (DPPC: CHOL (3:2)) and 15 mg of fasudil were dissolved in 10 mL organic solvent (Methanol:chloroform (2:1)). The resulting solution was rotary evaporated for 45 minutes to form a film. The film was hydrated with 2 mL 0.1 N NaOH. The hydrated film was bath sonicated for 5 minutes and probe sonicated for 6 minutes The liposomal composition was centrifuged at 15 k rpm for 15 minutes remove free fasudil. Three freeze thaw cycles were then performed (1 cycle=liquid nitrogen for 3 minutes and 45° C. water bath for 3 minutes).

### Example 19

For formulation F46, liposome formation was performed in the same manner as formulation F45 but using 40 mM lipids instead of 30 mM lipids.

### Example 20

Preparation of fasudil-loaded liposomes was performed according to the following procedure for formulation F47 and subsequent formulations:
1. Accurate amounts of 146 mg of DPPC (Dipalmitoylphosphatidylcholine) and 73 mg cholesterol were weighed in the ratio of 1:1 to prepare a final lipid concentration of 40 mM (w/w).
2. Lipids were dissolved by adding them in a vial or beaker and 7 mL of chloroform and 3 mL of methanol were added. The resulting solution was mixed until a clear solution was formed.
3. To the clear solution, 30 mg of fasudil was added.
4. The resulting solution was added to a round bottom flask and subjected to rotary evaporation under reduced pressure of 200 mm Hg. The stirring speed was maintained at 48 rpm and the water bath temperature was set at 60° C.
5. A thin film was allowed to form for about 45 minutes to about 1 hour.
6. After thin film formation, 2 ml of 0.1N NaOH solution was added to the round bottom flask.
7. Rotary evaporation was continued for another 2 hours (without pressure) under constant temperature.
8. A milky solution became visible during that additional period of rotary evaporation.
9. After 2 hours, the opening of the round bottom flask was sealed, and the flask was bath sonicated for 10-20 minutes to ensure complete removal of film from the surface of the flask.
10. To reduce the particle size of liposomes, probe sonication was performed for 6 minutes (10 seconds on and 10 seconds off).
11. The liposomes were collected and centrifuged at 15,000 rpm for 15 minutes to remove the unentrapped drug.
12. The liposomes were again collected in a cryovial to perform the freeze thaw method.
13. Cryovials were kept in liquid nitrogen for 3 minutes followed by incubating the vials in water bath at 40° C. This cycle was repeated 3 times and helped stabilize the liposomes by disrupting any quick assembly of lipid structures.

### Example 21

An additional step was performed, for example, for F58, F68, and F69, when preparing liposomes containing HPMC, or pegylated-DSPE, or both. For such formulations, HPMC, pegylated-DSPE, or both were dissolved in the organic solvent. For HPMC liposomes, 100 mg of HPMC NF was weighed and added to the clear solution.

### Example 22

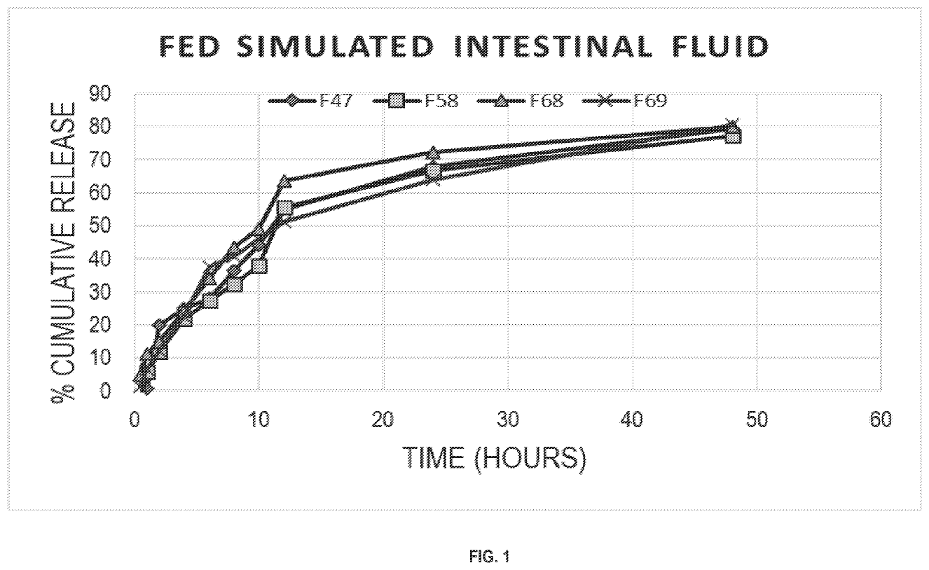

Formulations F47, F58, F68, and F69 were tested for release of fasudil from liposomes in FED simulated intestinal fluid. F47-base, F58-HPMC, F68-DSPE, F69-Pegylated-DSPE/HPMC, HPMC 100 mM, Pegylated-DSPE 0.5 mM, DPPC/Chol 40 mM All four formulations showed similar release profiles as depicted in FIG. 1.

The present disclosure includes the following aspects/embodiments/features in any order and/or in any combination:
1. An oral pharmaceutical composition comprising a liposome, the liposome comprising fasudil, a pharmaceutically acceptable salt thereof, a hydrate thereof, a prodrug thereof, a substituted derivative thereof, or a metabolite thereof, or any combination thereof.
2. The oral pharmaceutical composition of any preceding or following embodiment/feature/aspect, wherein the liposome comprises a single lamellar vesicle.
3. The oral pharmaceutical composition any preceding claim, wherein the liposome comprises a phospholipid and cholesterol.
4. The oral pharmaceutical composition of any preceding or following embodiment/feature/aspect, wherein the liposome comprises a monoacyl glyceride and cholesterol.
5. The oral pharmaceutical composition of any preceding or following embodiment/feature/aspect, wherein the liposome comprises a diacyl glyceride and cholesterol.
6. The oral pharmaceutical composition of any preceding or following embodiment/feature/aspect, wherein the liposome comprises the diacyl glyceride and cholesterol in a ratio of from about 0.1:1.0 to about 1.0:0.1.
7. The oral pharmaceutical composition of any preceding or following embodiment/feature/aspect, wherein the ratio is about 1:1.
8. The oral composition of any preceding or following embodiment/feature/aspect, wherein an esterified fatty acid of the diacyl glyceride comprises a saturated fatty acid, or an unsaturated fatty acid, or both.
9. The oral composition of any preceding or following embodiment/feature/aspect, wherein the unsaturated fatty acid comprises a monounsaturated fatty acid, or a polyunsaturated acid, or both.
10. The oral composition of any preceding or following embodiment/feature/aspect, wherein an esterified fatty acid of the diacyl glyceride comprises capric acid, lauric acid, myristic acid, palmitic acid, stearic acid, arachidic acid, behenic acid, or lignoceric acid, or any combination thereof.
11. The oral pharmaceutical composition of any preceding or following embodiment/feature/aspect, wherein an esterified fatty acid of the diacyl glyceride comprises alpha-linolenic acid, gamma-linolenic acid, stearidonic acid, eicosenoic acid, eicosapentaenoic acid, cervonic acid, linoleic acid, linolelaidic acid, arachidonic acid, palmitoleic acid, vaccenic acid, paullinic acid, oleic acid, elaidic acid, gondonic acid, erucic acid, nervonic acid, or mead acid, or any combination thereof.
12. The oral pharmaceutical composition of any preceding or following embodiment/feature/aspect, wherein the diacyl glyceride comprises a phospholipid, or a pegylated-phospholipid, or both.
13. The oral pharmaceutical composition of any preceding or following embodiment/feature/aspect, wherein the phospholipid comprises phosphatidylcholine, phosphatidylethanolamine, phosphatidylserine, phosphatidylglycerol, or phosphatidylinositol, or any combination thereof.
14. The oral pharmaceutical composition of any preceding or following embodiment/feature/aspect, wherein the phospholipid comprises dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoylphosphatidylethanolamine (DSPE), distearoylphosphatidylglycerol (DSPG), distearoylphosphatidylserine (DSPS), palmitoyloleoylphyosphatidylcholine (POPC), palmitoyloleoylphyosphatidylethanolamine (POPE), palmitoyloleoylphyosphatidylglycerol (POPG), palmitoyloleoylphyosphatidylserine (POPS), dioleoylphosphatidylethanolamine (DOPE), dioleoylphosphatidylserine (DOPS), dimyristoylphosphatidylcholine (DMPC), dimyristoylphosphatidylethanolamine (DMPE), dimyristoylphosphatidylglycerol (DMPG), or dimyristoylphosphatidylserine (DMPS), or any combination thereof.
15. The oral pharmaceutical composition of any preceding or following embodiment/feature/aspect, wherein the oral pharmaceutical composition is formulated as a solid dosage form.
16. The oral pharmaceutical composition of any preceding or following embodiment/feature/aspect, wherein the solid dosage form comprises a tablet, or a capsule, or both.
17. The oral pharmaceutical composition of any preceding or following embodiment/feature/aspect, wherein the oral pharmaceutical composition comprises an aqueous composition.
18. The oral pharmaceutical composition of any preceding or following embodiment/feature/aspect, wherein the aqueous composition comprises a humectant, a chelator, an antioxidant, or a preservative, or any combination thereof.
19. The oral pharmaceutical composition of any preceding or following embodiment/feature/aspect, wherein the aqueous composition comprises a solution, a suspension, an emulsion, a gel, or a colloid, or any combination thereof.
20. The oral pharmaceutical composition of any preceding or following embodiment/feature/aspect, wherein the aqueous composition further comprises a buffering agent.
21. The oral pharmaceutical composition of any preceding or following embodiment/feature/aspect, wherein the aqueous composition has a pH of from about 5.5 to about 7.5.
22. The oral pharmaceutical composition of any preceding or following embodiment/feature/aspect, wherein the liposome is sufficiently stable in an oral cavity to mask a bitter taste of the fasudil.
23. The oral pharmaceutical composition of any preceding or following embodiment/feature/aspect, wherein the liposome comprises a coating.
24. The oral pharmaceutical composition of any preceding or following embodiment/feature/aspect, wherein the coating comprises an enteric coating.
25. The oral pharmaceutical composition of any preceding or following embodiment/feature/aspect, wherein the coating is from about 0.1 w/w % to about 10 w/w % of the liposome.
26. The oral pharmaceutical composition of any preceding or following embodiment/feature/aspect, wherein the coating comprises a cellulose ether, or a polymethacrylate, or both.
27. The oral pharmaceutical composition of any preceding or following embodiment/feature/aspect, wherein the cellulose ether comprises hydroxypropylmethyl cellulose (HPMC; hypromellose).
28. The oral pharmaceutical composition of any preceding or following embodiment/feature/aspect, wherein the polymethacrylate comprises a polymethacrylate having a methacrylic acid:methyl methacrylate ratio of 1:1, or 1:2, or both.
29. The oral pharmaceutical composition of any preceding or following embodiment/feature/aspect, wherein the composition comprises a plurality of liposomes, the plurality of liposomes comprises the liposome, and at least 50% of liposomes in the plurality comprise fasudil, a pharmaceutically acceptable salt thereof, a hydrate thereof, a prodrug thereof, a substituted derivative thereof, or a metabolite thereof, or any combination thereof.
30. The oral pharmaceutical composition of any preceding or following embodiment/feature/aspect, wherein the concentration of fasudil, a pharmaceutically acceptable salt thereof, a hydrate thereof, a prodrug thereof, a substituted derivative thereof, or a metabolite thereof, or any combination thereof is from about 0.05 mg/mL to about 50 mg/mL.
31. The oral pharmaceutical composition of any preceding or following embodiment/feature/aspect, wherein the concentration of fasudil, a pharmaceutically acceptable salt thereof, a hydrate thereof, a prodrug thereof, a substituted derivative thereof, or a metabolite thereof, or any combination thereof is from about 5.0 mg/ml to about 20 mg/mL.
32. The oral pharmaceutical composition of any preceding or following embodiment/feature/aspect, wherein the fasudil is present in an amount from about 0.5% w/v to about 5.0% w/v of the aqueous composition.
33. The oral pharmaceutical composition of any preceding or following embodiment/feature/aspect, wherein the composition has a polydispersity index (PDI) less than about 1.0.
34. The oral pharmaceutical composition of any preceding or following embodiment/feature/aspect, wherein the composition comprises a plurality of liposomes, the plurality of liposomes comprises the liposome, and the mean diameter of the plurality of liposomes is from about 1.0 nm to about 500 μm.
35. The oral pharmaceutical composition of any preceding or following embodiment/feature/aspect, wherein the composition comprises a plurality of liposomes, the plurality of liposomes comprises the liposome, and the mean zeta value of the plurality of liposomes is from about −50 mv to about −5 mv.
36. The oral pharmaceutical composition of any preceding or following embodiment/feature/aspect, wherein the composition comprises a plurality of liposomes, the plurality of liposomes comprises the liposome, and the plurality comprises a small unilamellar vesicle, a large unilamellar vesicle, a giant unilamellar vesicle, a multilamellar vesicle, or multivesicular vesicle, or any combination thereof.
37. The oral pharmaceutical composition of any preceding or following embodiment/feature/aspect, wherein the composition comprises a plurality of liposomes, the plurality of liposomes comprises the liposome, and the liposomes of the plurality are present in the aqueous composition at a concentration of from about 1.0% w/v to about 80% w/V.
38. The oral pharmaceutical composition of any preceding or following embodiment/feature/aspect, wherein the composition comprises a plurality of liposomes, the plurality of liposomes comprises the liposome, and the fasudil, a pharmaceutically acceptable salt thereof, a hydrate thereof, a prodrug thereof, a substituted derivative thereof, or a metabolite thereof, or any combination thereof is present in the composition in an amount sufficient to treat a neurodegenerative disease.
39. The oral pharmaceutical composition of any preceding or following embodiment/feature/aspect, wherein the neurodegenerative disease comprises Alzheimer's disease, vascular dementia, amyotrophic lateral sclerosis (ALS), Parkinson's disease, Huntington's disease, multiple sclerosis, progressive supranuclear palsy (PSP), or corticobasal syndrome (CBS), or any combination thereof.
40. A method of treating a neurodegenerative disease comprising administering to a patient the composition of any preceding or following embodiment/feature/aspect in an amount sufficient to treat the neurogenerative disease.
41. The method of any preceding or following embodiment/feature/aspect, further comprising diagnosing the neurodegenerative disease prior to administering the composition to the patient.
42. The method of any preceding or following embodiment/feature/aspect, wherein treating the neurogenerative disease comprises ameliorating a symptom of the neurodegenerative disease.
43. The method of any preceding or following embodiment/feature/aspect, wherein the symptom comprises wandering.
44. A method of treating a neurological disorder, the method comprising orally administering to a subject a composition comprising a liposome, the liposome comprising fasudil, a pharmaceutically acceptable salt thereof, a hydrate thereof, a prodrug thereof, a substituted derivative thereof, or a metabolite thereof, or any combination thereof.
45. A method of forming fasudil-comprising liposomes formulated for oral administration, the method comprising:
   mixing a diacylglyceride, cholesterol, a rho kinase inhibitor, and, optionally, a cellulose ether in a volatile organic solvent to produce a first composition, the rho kinase inhibitor comprising fasudil, a pharmaceutically acceptable salt thereof, a hydrate thereof, a prodrug thereof, a substituted derivative thereof, or a metabolite thereof, or any combination thereof;
   subjecting the first mixture to evaporation to produce a second composition; applying an aqueous solution to the second composition to form a third composition; and
   imparting energy to the third composition to produce a fourth composition, the fourth composition comprising the fasudil-comprising liposomes formulated for oral administration.
46. The method of any preceding or following embodiment/feature/aspect, wherein the volatile organic solvent comprises chloroform, or methanol, or both.
47. The method of any preceding or following embodiment/feature/aspect, wherein the evaporation is performed below ambient pressure, or above ambient temperature, or both.
48. The method of any preceding or following embodiment/feature/aspect, wherein the aqueous solution comprises an alkaline solution.
49. The method of any preceding or following embodiment/feature/aspect, wherein the energy is imparted using sonication.
50. A method of forming fasudil-comprising enteric coated liposomes, the method comprising:
   producing fasudil-comprising liposomes comprising fasudil, a pharmaceutically acceptable salt thereof, a hydrate thereof, a prodrug thereof, a substituted derivative thereof, or a metabolite thereof, or any combination thereof; and
   coating the fasudil-comprising liposomes to form the fasudil-comprising enteric coated liposomes.

The present disclosure can include any combination of these various features or embodiments above and/or below as set forth in sentences and/or paragraphs. Any combination of disclosed features herein is considered part of the present disclosure. Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. The scope of the disclosure is not limited to the specific values recited when defining a range.

What is claimed is:

1. An oral pharmaceutical composition comprising a liposome, the liposome comprising fasudil, a pharmaceutically acceptable salt thereof, a hydrate thereof, a prodrug thereof, a substituted derivative thereof, or a metabolite thereof, or any combination thereof.

2. The oral pharmaceutical composition of claim 1, wherein the liposome comprises a phospholipid and cholesterol.

3. The oral composition of claim 2, wherein an esterified fatty acid of the phospholipid comprises a saturated fatty acid, or an unsaturated fatty acid, or both.

4. The oral composition of claim 2, wherein an esterified fatty acid of the phospholipid comprises capric acid, lauric acid, myristic acid, palmitic acid, stearic acid, arachidic acid, behenic acid, or lignoceric acid, or any combination thereof.

5. The oral pharmaceutical composition of claim 2, wherein the phospholipid comprises phosphatidylcholine, phosphatidylethanolamine, phosphatidylserine, phosphatidylglycerol, or phosphatidylinositol, or any combination thereof.

6. The oral pharmaceutical composition of claim 2, wherein the phospholipid comprises dipalmitoylphosphatidylcholine (DPPC), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoylphosphatidylethanolamine (DSPE), distearoylphosphatidylglycerol (DSPG), distearoylphosphatidylserine (DSPS), palmitoyloleoylphyosphatidylcholine (POPC), palmitoyloleoylphyosphatidylethanolamine (POPE), palmitoyloleoylphyosphatidylglycerol (POPG), palmitoyloleoylphyosphatidylserine (POPS), dioleoylphosphatidylethanolamine (DOPE), dioleoylphosphatidylserine (DOPS), dimyristoylphosphatidylcholine (DMPC), dimyristoylphosphatidylethanolamine (DMPE), dimyristoylphosphatidylglycerol (DMPG), or dimyristoylphosphatidylserine (DMPS), or any combination thereof.

7. The oral pharmaceutical composition of claim 1, wherein the oral pharmaceutical composition comprises an aqueous composition.

8. The oral pharmaceutical composition of claim 1, wherein the aqueous composition comprises a solution, a suspension, an emulsion, a gel, or a colloid, or any combination thereof.

9. The oral pharmaceutical composition of claim 1, wherein the liposome is sufficiently stable in an oral cavity to mask a bitter taste of the fasudil.

10. The oral pharmaceutical composition of claim 1, wherein the liposome comprises a coating.

11. The oral pharmaceutical composition of claim 10, wherein the coating comprises a cellulose ether, or a polymethacrylate, or both.

12. The oral pharmaceutical composition of claim 1, wherein the concentration of fasudil, a pharmaceutically acceptable salt thereof, a hydrate thereof, a prodrug thereof, a substituted derivative thereof, or a metabolite thereof, or any combination thereof is from about 0.05 mg/mL to about 50 mg/mL.

13. The oral pharmaceutical composition of claim 1, wherein the composition comprises a plurality of liposomes, the plurality of liposomes comprises the liposome, and the fasudil, a pharmaceutically acceptable salt thereof, a hydrate thereof, a prodrug thereof, a substituted derivative thereof, or a metabolite thereof, or any combination thereof is present in the composition in an amount sufficient to treat a neurodegenerative disease.

14. The oral pharmaceutical composition of claim 13, wherein the neurodegenerative disease comprises Alzheimer's disease, vascular dementia, amyotrophic lateral sclerosis (ALS), Parkinson's disease, Huntington's disease, multiple sclerosis, progressive supranuclear palsy (PSP), or corticobasal syndrome (CBS), or any combination thereof.

15. A method of treating a neurodegenerative disease comprising administering to a patient the composition of claim 1 in an amount sufficient to treat the neurogenerative disease.

16. The method of claim 15, wherein treating the neurogenerative disease comprises ameliorating a symptom of the neurodegenerative disease.

17. The method of claim 16, wherein the symptom comprises wandering.

18. (canceled)

19. A method of forming fasudil-comprising liposomes formulated for oral administration, the method comprising:
mixing a diacylglyceride, cholesterol, a rho kinase inhibitor, and, optionally, a cellulose ether in a volatile organic solvent to produce a first composition, the rho kinase inhibitor comprising fasudil, a pharmaceutically acceptable salt thereof, a hydrate thereof, a prodrug thereof, a substituted derivative thereof, or a metabolite thereof, or any combination thereof;
subjecting the first mixture to evaporation to produce a second composition;
applying an aqueous solution to the second composition to form a third composition; and
imparting energy to the third composition to produce a fourth composition, the fourth composition comprising the fasudil-comprising liposomes formulated for oral administration.

20. A method of forming fasudil-comprising enteric coated liposomes, the method comprising:
producing fasudil-comprising liposomes comprising fasudil, a pharmaceutically acceptable salt thereof, a hydrate thereof, a prodrug thereof, a substituted derivative thereof, or a metabolite thereof, or any combination thereof; and
coating the fasudil-comprising liposomes to form the fasudil-comprising enteric coated liposomes.
